SEQUENCE LISTING (3 sequences)

SEQ ID NO: 1 - protein, 405 aa, Homo sapiens
MQEKDASSQGFLPHFQHFATQAIHVGQDPEQWTSRAVVPPISLSTTFKQGAPGQHSGFEYSRSGNPTRNCLEKAVAALDGAKYCLAFASGLAATVTITHLLKAGDQIICMDDVYGGTNRYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMEKHFKNGMAVAQFLESNPWVEKVIYPGLPSHPQHELVKRQCTGCTGMVTFYIKGTLQHAEIFLKNLKLFTLAESLGGFESLAELPAIMTHASVLKNDRDVLGISDTLIRLSVGLEDEEDLLEDLDQALKAAHPPSGSHS

SEQ ID NO: 2 - protein, 405 aa, Artificial Sequence
  MODIFIED CYSTATHIONINE-GAMMA-LYASE
MQEKDASSQGFLPHFQHFATQAIHVGQDPEQWTSRAVVPPISLSTTFKQGAPGQHSGFIYSRLGNPTRNCLEKAVAALDGAKYCLAFASGMAATVTITHLLKAGDQIICMDDVYGGTNDYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMERHFKNGMAVAQFLESNPWVEKVIYPGLPSHPQHELVKRQCTGCGGMVTFYIKGTLQHAEIFLKNLKLFTLAVSLGGFESLAELPASMTHASVLKNDRDVLGISDTLIRLSVGLEDEEDLLEDLDQALKAAHPPSGSHS

SEQ ID NO: 3 - protein, 404 aa, Artificial Sequence
  MODIFIED
QEKDASSQGFLPHFQHFATQAIHVGQDPEQWTSRAVVPPISLSTTFKQGAPGQHSGFIYSRLGNPTRNCLEKAVAALDGAKYCLAFASGMAATVTITHLLKAGDQIICMDDVYGGTNDYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMERHFKNGMAVAQFLESNPWVEKVIYPGLPSHPQHELVKRQCTGCGGMVTFYIKGTLQHAEIFLKNLKLFTLAVSLGGFESLAELPASMTHASVLKNDRDVLGISDTLIRLSVGLEDEEDLLEDLDQALKAAHPPSGSHS

(19) United States
(12) Patent Application Publication
(10) Pub. No.: US 2024/0058429 A1
(43) Pub. Date: Feb. 22, 2024

(54) Treatment Of Homocystinuria And Hyperhomocysteinemia Using Cystathionine-Gamma-Lyase

(71) Applicant: Aeglea Bio Therapeutics, Inc., Austin, TX (US)

(72) Inventors: Scott W. Rowlinson, Austin, TX (US); Leslie Sloan, Austin, TX (US); Christopher Daige, Austin, TX (US)

(73) Assignee: Aeglea Bio Therapeutics, Inc., Austin, TX (US)

(21) Appl. No.: 18/000,183

(22) PCT Filed: May 29, 2020
(86) PCT No.: PCT/US2020/035297

§ 371 Date: Nov. 29, 2022

(51) Int. Cl. A61K 38/51; A61K 47/60; A61P 3/00

(52) U.S. Cl. CPC A61K 38/51; A61K 47/60; A61P 3/00

(57) ABSTRACT

The present disclosure generally relates to methods for treating homocystinuria and hyperhomocysteinemia patients with cystathionine-gamma-lyase (CGL) enzymes.

### TECHNICAL FIELD

The present disclosure generally relates to methods for treating homocystinuria and hyperhomocysteinemia patients.

### BACKGROUND

Homocystinuria is an autosomal recessively inherited disorder of methionine metabolism that leads to a broad range of serious consequences with irreversible morbidity and reduced life expectancy due to elevated homocysteine and homocysteine metabolites (homocystine, homocysteine-cysteine complex, and others) in plasma and urine. Homocystinuria is an inherited defect in the transsulfuration pathway (homocystinuria I) or methylation pathway (homocystinuria II and III). In addition to increased plasma levels of homocysteine and homocystine, affected subjects have increased plasma levels of methionine and S-adenosylhomocysteine, and decreased levels of plasma cystathionine and cysteine. The main clinical features include: developmentally delayed learning difficulties, and intellectual disability; skeletal abnormalities (e.g., excessive height, long narrow limbs [dolichostenomelia], scoliosis, pectus excavatum, marfanoid appearance); Ectopia lentis (dislocation of the ocular lens), glaucoma, and/or severe myopia; premature thromboembolic vascular events; and fatty liver.

Normally, homocysteine is metabolized through the transsulfuration pathway into cystathionine and ultimately cysteine or homocysteine may be converted back to methionine by the remethylation pathway (FIG. 1). Homocystinuria is caused by mutation of one of the genes related to the transsulfuration or remethylation pathways, including: cystathionine β-synthase (CBS), metabolism of cobalamin (cbl) associated D (MMADHC); 5,10-methylenetetrahydrofolate reductase (MTHFR); 5-methyltetrahydrofolate-homocysteine methyltransferase (MTR); or 5-methyltetrahydrofolate-homocysteine methyltransferase reductase (MTRR).

Homocystinuria patients are unable to process certain building blocks of proteins (amino acids) properly. There are multiple forms of homocystinuria, which are distinguished by their signs and symptoms and genetic cause. Homocystinuria due to mutations in the CBS gene, also known as classical homocystinuria (OMIM 236200), is the most common form of homocystinuria. The function of CBS enzyme is to convert homocysteine to cysthathionine (first step of the transsulfuration pathway), but defects or deficiencies of CBS lead to accumulation of homocysteine, which can also lead to accumulation of the amino acid methionine due to remethylation of homocysteine. Individuals with the common form of homocystinuria are characterized by nearsightedness (myopia), dislocation of the lens at the front of the eye, an increased risk of abnormal blood clotting, and brittle bones that are prone to fracture (osteoporosis) or other skeletal abnormalities. Some affected individuals also have developmental delay and learning problems.

More rarely, homocystinuria can be caused by mutations in the MTHFR, MTR, MTRR, and MMADHC genes. Enzymes encoded by these genes play a role in converting homocysteine to methionine but mutations will lead to a buildup of homocysteine in the body. Individuals can present with intellectual disability, failure to grow and gain weight at the expected rate (failure to thrive), seizures, problems with movement, and a blood disorder known as megaloblastic anemia. Megaloblastic anemia occurs when a person has a low number of red blood cells (anemia), and the remaining red blood cells are larger than normal (megaloblastic). The signs and symptoms of homocystinuria typically develop within the first year of life, although some mildly affected people may not develop features until later in childhood or adulthood.

The well-characterized natural history of homocystinuria due to CBS deficiency demonstrates that the devastating complications of the disease are strongly associated with chronic exposure to elevated levels of plasma total homocysteine (tHcy). Previous studies demonstrate that lowering of plasma tHcy has a consistent and favorable impact to reduce disease-related complications. Thus, the emphasis of expert-defined treatment guidelines on the primary treatment objective of homocystinuria due to CBS deficiency is to lower plasma tHcy.

Patients with homocystinuria due to CBS deficiency are known to have cognitive, behavioral, and psychiatric disease-related manifestations that profoundly impact the patients and their families. Many homocystinuria patients with CBS deficiency have psychiatric disorders and chronic behavioral disorders, some of which may not conform to standard diagnostic categories. Reducing homocysteine levels through disease management is believed to improve behavior and even intellectual ability. Therefore, managing adaptive behavior is potentially of clinical relevance in subjects with homocystinuria due to CBS deficiency, and it is reasonable to hypothesize that earlier treatment to reduce plasma tHcy may lead to improvements in adaptive behavior.

The current disease management of subjects with homocystinuria due to CBS deficiency includes restriction of dietary protein and methionine with the goal to lower the plasma tHcy concentration to a recommended level while maintaining adequate nutrition. Frequent metabolic monitoring with plasma amino acid analysis is required to assess response to diet. In addition, the degree of protein restriction in the diet of subjects with homocystinuria due to CBS deficiency is challenging to maintain and compliance is poor. Furthermore, this degree of dietary protein restriction can contribute to long-term nutritional deficiencies and social acceptance challenges, which contribute to noncompliance and the overall disease burden. Adherence to a strict amino acid restricted diet is challenging for many subjects with homocystinuria. A treatment option that allows patients to have liberalization in dietary restrictions would provide a major advance in the quality of life. Therefore, patients with dietary liberalization will be able to increase natural protein intake and reduce or eliminate medical foods and amino acid supplements when they are able to biochemically control and maintain reduced plasma tHcy levels.

The unmet medical need for patients with homocystinuria due to CBS-deficiency is in three main areas. First, therapy-resistant patients on optimal standard disease management who are unable to consistently maintain tHcy levels below desired target concentrations. Second, patients who are at high risk of non-compliance with a low methionine diet and/or adjunctive therapies (pyridoxine and/or betaine therapy), which places them at increased risk of severe complications including sudden death (e.g., older teenagers and young adults or patients with behavioral problems or intellectual disability). Third, patients intolerant of available adjunctive therapies, due to either safety issues or other adverse reactions who have tHcy levels above the desired target concentrations.

Current therapies are significantly limited by non-compliance and limited effectiveness in controlling plasma tHcy. There is a clear unmet need for an alternative therapy that would provide better control of plasma tHcy and allow for diet liberalization and discontinuation of betaine. Such a therapy would minimize the chronic, lifelong exposure to toxic levels of plasma tHcy, thus reducing the serious consequences of homocystinuria and burden on patients and their caregivers.

### SUMMARY

One aspect of the present invention relates to a method of treating a subject having or at risk of developing homocystinuria or hyperhomocysteinemia. In one or more embodiments of this aspect, the method comprises administering a therapeutically effective amount of formulation comprising a modified human cystathionine-γ-lyase (CGL) enzyme comprising at least the following substitutions relative to a native human CGL amino acid sequence (SEQ ID NO: 1): isoleucine at position 59, leucine at position 63, methionine at position 91, aspartic acid at position 119, arginine at position 268, glycine at position 311, valine at position 339, and serine at position 353. In one or more embodiments, the formulation is administered at an enzyme dose of about 0.05 mg/kg to about 4 mg/kg at a frequency of one dose per day to one dose per month.

In one or more embodiments, enzyme is bound to one or more PEG units.

In one or more embodiments, the formulation further comprises a pharmaceutically acceptable carrier.

In one or more embodiments, the formulation is 2 mL of a liquid supplied in a 5 mL vial.

In one or more embodiments, wherein the formulation comprises an enzyme concentration of about 1 mg/mL to about 50 mg/mL. In one or more embodiments, the formulation comprises an enzyme concentration of about 1 to about 20 mg/mL.

In one or more embodiments, the formulation is administered intravenously or subcutaneously.

In one or more embodiments, the formulation is administered one dose per week.

In one or more embodiments, the formulation is administered intravenously for four weeks, followed by subcutaneous administration in subsequent weeks.

In one or more embodiments, the formulation is administered to the subject at a dose of about 0.1 mg/kg to about 1 mg/kg. In one or more embodiments, the formulation is administered once a week at a dose of about 0.1 mg/kg to about 1 mg/kg. In one or more embodiments, the formulation is administered to the subject at a dose of about 0.15 mg/kg, about 0.45 mg/kg, or about 1 mg/kg.

In one or more embodiments, the formulation is diluted in saline prior to intravenously administering to the subject.

In one or more embodiments, the subject is a human patient.

In one or more embodiments, the subject has a total plasma homocysteine level greater than 80 μM prior to initiating therapy with administering the formulation.

In one or more embodiments, the subject is at least 12 years of age.

In one or more embodiments, the subject is maintained on an individualized diet.

In one or more embodiments, the subject is maintained on a methionine-restricted diet.

In one or more embodiments, the method reduces total plasma homocysteine levels. In one or more embodiments, the method reduces total plasma homocysteine levels to less than or equal to about 80 VM. In one or more embodiments, method reduces total plasma homocysteine levels to less than or equal to about 50 μM. In one or more embodiments, the method reduces total plasma homocysteine levels to less than or equal to about 15 μM.

### BRIEF DESCRIPTION OF THE DRAWINGS

Further features of the present invention will become apparent from the following written description and the accompanying figures, in which:

FIG. 1 graphically represents the Methionine Remethylation Pathway in which homocysteine can be converted back to methionine by the remethylation pathway or homocysteine can be metabolized to cystathionine by the transsulfuration pathway.

FIG. 2A shows the primary amino acid sequence of human cystathionine-γ-lyase (CGL) (SEQ ID NO: 1), FIG. 2B shows the primary amino acid sequence of a modified human cystathionine-γ-lyase with the mutations E591, S63L, L91M, R119D, K268R, T311G, E339V, and I353S (CGL-ILMDRGVS) (SEQ ID NO: 2) and FIG. 2C shows the primary amino acid sequence of a modified human cystathionine-γ-lyase lacking a N-terminal methionine and with the mutations E591, S63L, L91M, R119D, K268R, T311G, E339V, and I353S (CGL-ILMDRGVS-ΔM) (SEQ ID NO: 3).

FIG. 3 graphically represents a study schema for a first-in-human, open-label, non-randomized Phase 1/2 multiple-ascending-dose study composed of two parts: a single IV cohort of 4 subjects in Part 1 who will receive 4 QW doses of study drug followed by 3 planned SC cohorts of 4 subjects each in Part 2 who will receive 4 QW doses of study drug.

FIG. 4 graphically represents a Kaplan Meier Curve for survival of CBS-deficient (CBS−/−) mice treated with HIS-CGL-ILMDRGVS-PEG.

FIG. 5 graphically represents a Kaplan Meier Curve for survival of CBS-deficient (CBS−/−) mice treated with CGL-ILMDRGVS-PEG.

FIGS. 6A-6E graphically represent liver samples and representative H&E staining from each liver. FIG. 6A shows healthy liver from a wild-type mouse. FIG. 6B shows 10-day old untreated CBS−/− mouse. FIG. 6C shows 23-day old PBS treated CBS−/− mouse. FIG. 6D shows 23-day old HIS-CGL-ILMDRGVS-PEG treated CBS−/− mouse. FIG. 6E shows 60-day old HIS-CGL-ILMDRGVS-PEG treated CBS−/− mouse.

FIGS. 7A-7D graphically represent H&E stained liver samples. FIG. 7A shows 10-day old untreated CBS−/− mice. FIG. 7B shows 21-day old PBS treated CBS−/− mice. FIG. 7C shows 21-day old BIW 6.25 mg/kg polyHistidine-tagged and PEGylated CGL-ILMDRGVS (HIS-CGL-ILMDRGVS-PEG) treated CBS−/− mice. FIG. 7D shows 21-day old BIW 12.5 mg/kg HIS-CGL-ILMDRGVS-PEG treated CBS−/− mice.

FIGS. 8A-8D graphically represent Oil-Red-O stained liver samples. FIG. 8A shows 10-day old untreated CBS−/− mice. FIG. 8B shows 19 to 21-day old PBS treated CBS−/− mice FIG. 8C shows 20-day old TIW 1.56 mg/kg polyHistidine-tagged and PEGylated CGL-ILMDRGVS (HIS-CGL-ILMDRGVS-PEG) treated CBS−/− mice FIG. 8D shows 21-day old BIW 12.5 mg/kg HIS-CGL-ILMDRGVS-PEG treated CBS−/− mice.

FIGS. 9A and 9B graphically represent CBS−/− mice treated with 10 mg/kg CGL-ILMDRGVS-PEG in which FIG. 9A shows degree of hair loss (alopecia) on day 170 and FIG. 9B bone mineral density (BMD) to check for osteoporosis on day 169 were measured as mean±standard error of the mean (SEM). *p≤0.05 by Mann-Whitney test.

FIGS. 10A-10C graphically represents tHcy levels in plasma (FIG. 10A), liver (FIG. 10B), and brain (FIG. 10C) samples of CBS−/− mice following multiple subcutaneous (SC) BIW of CGL-ILMDRGVS-PEG. tHcy levels were measured as mean±standard error of the mean (SEM). *p<0.05 by Mann-Whitney test.

FIG. 11 graphically represents a single-dose pharmacodynamic study in CBS-deficient (CBS−/−) mice in which plasma total homocysteine (tHcy) levels following a single subcutaneous (SC) dose of 12.5 mg/kg or 6.25 mg/kg dose of Polyhistidine-tagged and PEGylated CGL-ILMDRGVS (HIS-CGL-ILMDRGVS-PEG) was measured over time.

FIG. 12 graphically represents a single-dose pharmacodynamic study in CBS-deficient (CBS−/−) mice in which plasma total methionine levels following a single subcutaneous (SC) dose of 12.5 mg/kg or 6.25 mg/kg dose of Polyhistidine-tagged and PEGylated CGL-ILMDRGVS (HIS-CGL-ILMDRGVS-PEG) was measured over time. A single mouse was excluded from the 12.5 mg/kg group.

FIG. 13 graphically represents a multi-dose pharmacodynamics study in CBS-deficient (CBS−/−) mice in which tHcy plasma levels following multiple doses of polyHistidine-tagged and PEGylated CGL-ILMDRGVS (HIS-CGL-ILMDRGVS-PEG) was measured over time.

FIGS. 14A and 14B graphically represent single-dose pharmacokinetics of PEGylated CGL-ILMDRGVS intravenously administered to Cynomolgus Monkeys. FIG. 14A is a linear graphical representation and FIG. 14B is a semi-log graphical representation.

FIGS. 15A and 15B graphically represent multi-dose pharmacokinetics of PEGylated CGL-ILMDRGVS subcutaneously administered to Cynomolgus Monkeys at 8 mg/kg (1^{st} dose on day 1) and 2 mg/kg (2^{nd} dose on day 15). FIG. 15A is a linear graphical representation and FIG. 15B is a semi-log graphical representation.

FIGS. 16A and 16B graphically represent single-dose pharmacodynamics study in Cynomolgus Monkeys in which mean plasma tHcy concentration following intravenous administration of PEGylated CGL-ILMDRGVS were measured over time. FIG. 16A is a linear graphical representation and FIG. 16B is a semi-log graphical representation.

FIGS. 17A and 17B graphically represents multi-dose pharmacodynamics study in Cynomolgus Monkeys in which mean plasma tHcy concentration following subcutaneous administrations of 8 mg/kg (1^{st} dose on day 1) and 2 mg/kg (2^{nd} dose on day 15) were measured over time. FIG. 17A is a linear graphical representation and FIG. 17B is a semi-log graphical representation.

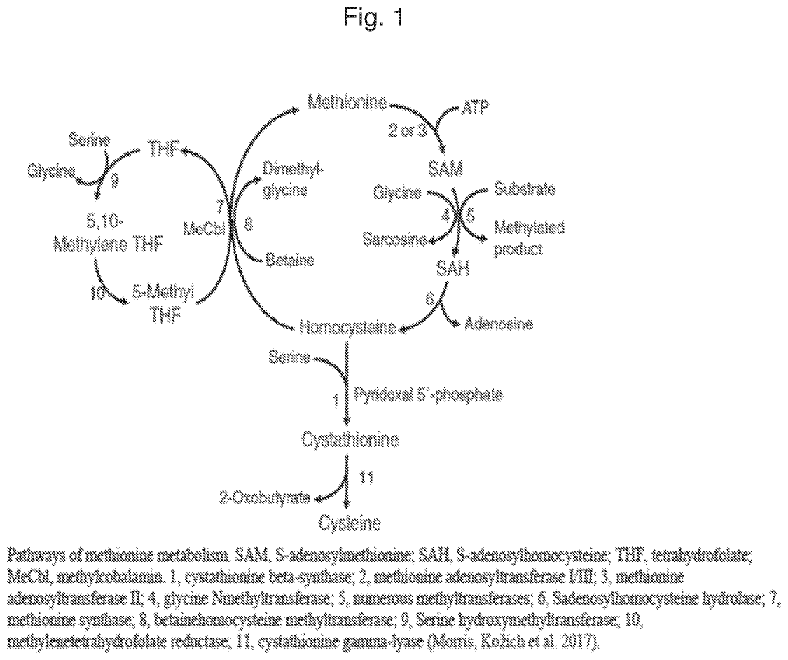

### DETAILED DESCRIPTION

There is an unmet medical need for patients with homocystinuria and hyperhomocysteinemia due to cystathionine β-synthase (CBS)-deficiency. An engineered human cystathionine γ-lyase (CGL) was designed to degrade homocysteine and homocystine. Modifications to CGL (e.g., PEGylation and/or Polyhistidine tag) and various mutations (e.g., E591, S63L, L91M, R119D, K268R, T311G, E339V, I353S) introduced to the CGL active site resulted in a molecule that has high substrate specificity for homocysteine and homocystine but not for the native substrate, cystathionine. For classical homocystinuria patients, the engineered enzyme is anticipated to ameliorate the adverse impact of CBS enzyme deficiency in the transsulfuration pathway by providing an alternate pathway for enzymatic degradation of high plasma total homocysteine levels (tHcy). Current therapies are significantly limited by non-compliance and limited effectiveness in controlling plasma tHcy. There is a clear unmet need for an alternative therapy that would provide better control of plasma tHcy and allow for diet liberalization and discontinuation of betaine. The engineered enzyme would be advantageous to minimize the chronic, lifelong exposure to toxic levels of plasma tHcy, thus reducing the serious consequences of homocystinuria (e.g., osteoporosis, neurological, and psychological complications, and thromboembolic events) and burden on patients and their caregivers.

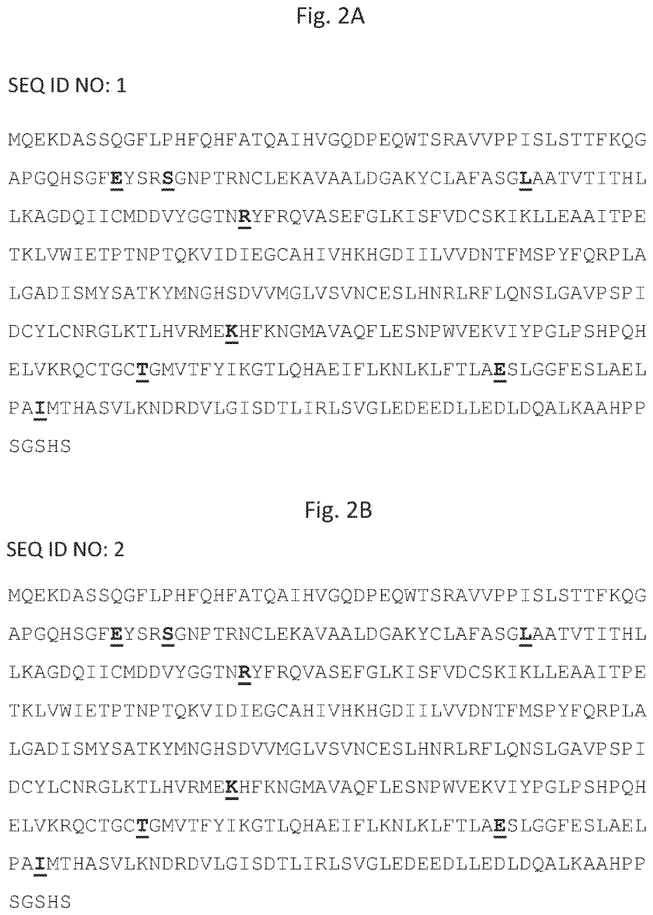

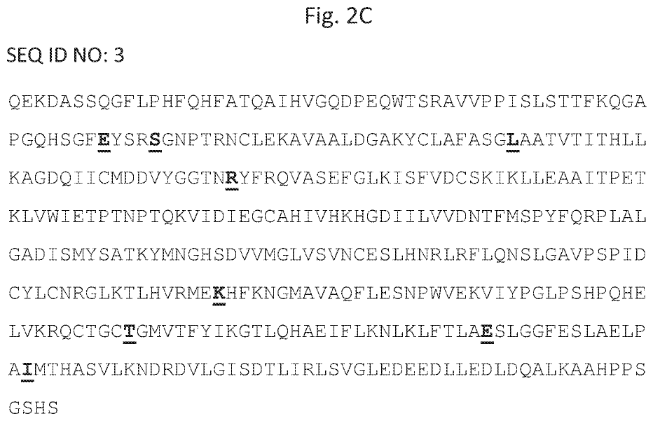

An engineered human cystathionine γ-lyase with mutations E591, S63L, L91M, R119D, K268R, T311G, E339V, I353S (CGL-ILMDRGVS) was developed as a treatment for homocystinuria and hyperhomocysteinemia. CGL-ILMDRGVS (and its PEGylated form: CGL-ILMDRGVS-PEG) is a pyridoxal phosphate (PLP)-dependent tetramer that catalyzes the conversion of homocysteine and homocystine to alpha ketobutyrate, thio-homocysteine, hydrogen sulfide and ammonia. CGL-ILMDRGVS breaks down also methionine and cysteine with ˜20 and ˜10-fold less potency than the preferred substrates homocysteine and homocystine. CGL-ILMDRGVS was developed via protein engineering of the human enzyme cystathionine γ-lyase (CGL, EC 4.4.1.1). PEGylation of CGL-ILMDRGVS yields the CGL-ILMDRGVS-PEG compound with an extended serum half-life. Like CGL, CGL-ILMDRGVS-PEG (and CGL-ILMDRGVS) forms a homotetramer by non-covalent bonds. Covalent binding of the co-factor pyridoxal phosphate (PLP) in the active site of CGL-ILMDRGVS-PEG is essential for its catalytic activity. The effects of cystathionine-gamma-lyase (CGL) variant enzymes on pharmacokinetics (PK) and pharmacodynamics (PD), including plasma total homocysteine (tHcy) levels, were determined to find an effective human dose to treat homocystinuria and hyperhomocysteinemia. The biochemical properties of CGL-ILMDRGVS are summarized in Table 1 and the primary amino acid sequence (including mutated amino acids) is shown in FIG. 2.

TABLE 1
Biochemical properties of CGL-ILMDRGVS (unPEGylated intermediate)

| | |
|---|---|
| Molecular weight of monomeric | 44,423 Daltons (calculated value) |
| CGL-ILMDRGVS | |
| liomotetrameric CGL-ILMDRGVS | 177,692 Daltons (calculated value) |
| Length of the monomer | 405 amino acid residues |
| Disulfide bonds: | None |

Accordingly, various embodiments of the present invention are directed to administration of modified CGL enzymes such as CGL-ILMDRGVS and CGL-ILMDRGVS-PEG.

### Definitions

As described herein the terms “enzyme” and “protein” and “polypeptide” refer to compounds comprising amino acids joined via peptide bonds and are used interchangeably.

As used herein, the term “fusion protein” refers to a chimeric protein containing proteins or protein fragments operably linked in a non-native way.

As described herein, the term “half-life” (½-life) refers to the time that would be required for the concentration of a polypeptide to fall by half in vitro (e.g., as measured in cell culture media) or in vivo (e.g., as measured in serum), for example, after injection in a mammal. Methods to measure “half-life” include the use of antibodies specific for CGL or PEG used in an ELISA (enzyme-linked immunosorbent assay) format such that the physical amount of protein is measured as a function of time. Other methods germane to measuring the half-life include determining the catalytic activity of the enzyme drug as a function of time by any assay that detects the production of any substrates resulting from conversion of homocyst(e)ine, to products such as alpha-ketobutyrate, methanethiol, and/or ammonia.

The term “linker” is meant to refer to a compound or moiety that acts as a molecular bridge to operably link two different molecules, wherein one portion of the linker is operably linked to a first molecule, and wherein another portion of the linker is operably linked to a second molecule.

As described herein, the term “PEGylated” refers to conjugation with polyethylene glycol (PEG), which has been widely used as a drug carrier, given its high degree of biocompatibility and ease of modification. PEG can be coupled (e.g., covalently linked) to active agents through the hydroxy groups at the end of the PEG chain via chemical methods; however, PEG itself is limited to at most two active agents per molecule. In a different approach, copolymers of PEG and amino acids have been explored as novel biomaterial that would retain the biocompatibility of PEG, but that would have the added advantage of numerous attachment points per molecule (thus providing greater drug loading), and that can be synthetically designed to suit a variety of applications.

As described herein, the term “gene” refers to a DNA sequence that comprises control and coding sequences necessary for the production of a polypeptide or precursor thereof. The polypeptide can be encoded by a full-length coding sequence or by any portion of the coding sequence so as the desired enzymatic activity is retained.

As described herein, the term “native” refers to the typical or wild-type form of a gene, a gene product, or a characteristic of that gene or gene product when isolated from a naturally occurring source. In contrast, the term “modified,” “variant,” “mutein,” or “mutant” refers to a gene or gene product that displays modification in sequence and functional properties (i.e., 25 altered characteristics) when compared to the native gene or gene product, wherein the modified gene or gene product is genetically engineered and not naturally present or occurring.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al, 1994, both incorporated herein by reference).

The term “expression vector” refers to any type of genetic construct comprising a nucleic acid coding capable of being transcribed to an RNA. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well (e.g. antibiotic resistance, multiple cloning site, etc.).

The term “therapeutically effective amount” as used herein refers to an amount of a therapeutic composition or formulation (e.g., a modified CGL enzyme or a nucleic acid encoding such an enzyme) that is employed in methods to achieve a therapeutic effect, i.e., to deplete homocyst(e)ine in a patient's circulation to a level that is at or below a normal reference value.

As described herein, the term “cystathionine-γ-lyase” (CGL or cystathionase) refers to any enzyme that catalyzes the hydrolysis of cystathionine to cysteine. As used herein, the terms also contemplate primate forms of cystathionine-γ-lyase (or cystathionine-gamma-lyase), including the human form of cystathionine-γ-lyase.

As described herein, the terms “treatment” and “treating” refer to administration or application of a therapeutic agent to a subject or performance of a procedure or modality on a subject for the purpose of obtaining a therapeutic benefit of a disease or health-related condition. For example, a treatment may include administration of a therapeutically effective amount of a homocyst(e)inase.

As described herein, the terms “subject” and “patient” refer to either a human or non-human, such as primates, mammals, and vertebrates. The subject may be a human.

Non-Clinical Dose Range Finding (DRF) Studies

Nonclinical experiments were performed using PEGylated cystathionine γ-lyase with mutations E591, S63L, L91M, R119D, K268R, T311G, E339V, I353S (CGL-ILMDRGVS-PEG) to characterize the pharmacology, PK, PD, safety, and toxicology of the compound. These studies support and enable the use of PEGylated CGL-ILMDRGVS (CGL-ILMDRGVS-PEG) in a Phase 1/2 first-in-human clinical study in subjects with homocystinuria due to CBS deficiency.

Deficiency of cystathionine β-synthase (CBS) causes elevated plasma levels of homocysteine and analogs of homocysteine (such as homocystine and homocysteinylated peptides) that, when combined, represent plasma total homocysteine (tHcy). In vivo animal studies have utilized the intraperitoneal (IP) and subcutaneous (SC) routes of administration to assess the pharmacologic effects of polyhistidine tagged CGL-ILMDRGVS-PEG (HIS-CGL-ILMDRGVS-PEG) on plasma tHcy levels in a clinically relevant knockout mouse model of classical homocystinuria.

In vivo animal studies were performed in cynomolgus monkeys which characterized CGL-ILMDRGVS-PEG Pharmacokinetic (PK), Pharmacodynamic (PD; tHcy depletion), and bioavailability utilizing both intravenous (IV) and subcutaneous (SC) routes of administration. These data were used to design the nonclinical safety and toxicology (toxicity, toxicokinetics, and immunogenic potential) of CGL-ILMDRGVS-PEG in normal Sprague Dawley rats and cynomolgus monkeys. Normal rats and monkeys are considered to provide a sensitive assessment of CGL-ILMDRGVS-PEG toxicology because they have low levels of plasma tHcy. This low plasma tHcy translating to a more marked depletion in their blood tHcy levels than expected in homocystinuria patients.

The nonclinical safety profile of CGL-ILMDRGVS-PEG used to support the clinical development plan also supported dose range finding (DRF) studies following multiple IV or SC CGL-ILMDRGVS-PEG doses in rats and cynomolgus monkeys and from planned 4-week Good Laboratory Practice (GLP) toxicology studies in the same species. Rats received IV or SC doses once weekly for 4 weeks starting on post-natal day 22, while cynomolgus monkeys (about 36 months of age) received IV or SC doses once weekly for 4 weeks. The 4-week toxicology studies will establish the no observed adverse effect level (NOAEL) needed to support the initiation of clinical dosing and study duration in subjects 12 years or older.

To enable clinical use of CGL-ILMDRGVS-PEG in pediatric patients younger than 12 years of age (≥2 to ≤11 years) toxicology was assessed in juvenile rats. The nonclinical studies with CGL-ILMDRGVS-PEG in the CBS−/− mouse disease model showed that administration of the HIS-CGL-ILMDRGVS-PEG can rescue neonatal lethality. The data generated from the HIS-CGL-ILMDRGVS-PEG and CGL-ILMDRGVS-PEG nonclinical studies provide information to support the first-in-human Phase 1/2 clinical study in subjects with homocystinuria due to CBS deficiency.

The planned and ongoing nonclinical studies in support of the IND and the overall nonclinical development plan are summarized in Table 2.

TABLE 2
CGL-ILMDRGVS-PEG Planned or Ongoing Nonclinical Studies

| | | | | Duration and | |
|---|---|---|---|---|---|
| | GLP | | Route of | Administration | Dose* |
| Study Type | status | Species | Administration | Schedule | mg/kg |
| Proof of Concept | Non- | CBS —/— | SC | Maintenance | HIS-CGL- |
| | GLP | mouse (D1) | | BIW 10 weeks | ILMDRGVS-PEG |
| | | | | Once | 3.125 to 25 |
| | | | | | mg/kg |
| | | | | | 12.5, 6.25 |
| | | | | | mg/kg |
| Proof of Concept | Non- | CBS —/— | SC | Maintenance | HIS-CGL- |
| | GLP | mouse (D1) | | BIW 4-8 weeks | ILMDRGVS-PEG |
| | | | | BIW | 12.5 mg/kg |
| | | | | QW | 12.5, 6.25 mg/kg |
| | | | | | 12.5 mg/kg |
| Repeat dose | Non- | CBS —/— | Subcutaneous | BIW | 1, 3, 10 mg/kg |
| | GLP | mouse (D1) | | 12 weeks | |
| Single and repeat | Non- | CBS —/— | Subcutaneous | Maintenance | HIS-CGL- |
| dose | GLP | mouse (D1) | | BIW 4-8 weeks | ILMDRGVS-PEG |
| | | | | Once | 10 mg/kg |
| | | | | Multiple | CGL- |
| | | | | | ILMDRGVS- |
| | | | | | PEG |
| | | | | | 1, 3, 10 mg/kg |
| Single and repeat | Non- | Cynomolgus | IV SC | Once in Phase 1 | 2, 8 mg/kg |
| dose | GLP | monkey | | Repeat SC in | 2 mg/kg |
| | | | | Phase 2 | |
| Dose range finding | Non- | Rat (early | SC | 4-week | 3, 10, 30 mg/kg |
| | GLP | adolescent, | | | |
| | | ~D45) | | | |
| Dose range finding | Non- | Cynomolgus | SC | 4-week | 1, 3, 10 mg/kg |
| | GLP | monkey (early | IV | | |
| | | adolescent, | | | |
| | | ~36 months) | | | |
| Repeat dose | GLP | Rat (early | SC | 4-week | 3, 10, 30 mg/kg |
| | | adolescent, | IV | | |
| | | ~D45) | | | |
| Repeat dose | GLP | Cynomolgus | SC | 4-week | 1, 3, 10 mg/kg |
| | | monkey (early | IV | | |
| | | adolescent, | | | |
| | | ~36 months) | | | |
| Repeat dose | GLP | Monkey | SC | 6-month | 1, 3, 10 mg/kg |
| Repeat dose | GLP | Rat (juvenile | SC | 6-month | 3, 10, 30 mg/kg |
| | | PND D21 to | | | |
| | | PND D168) | | | |
| Rcpcat dosc | GLP | Rat (juvenilc | SC | 6-wcck | 3, 10, 30 mg/kg |
| | | PND D21 to | | | |
| | | PND D63) | | | |
| Development and | GLP | Rat and | SC | TBD | TBD |
| Reproductive | | Rabbit | | | |
| Toxicology | | | | | |

Non-Clinical Pharmacology and Pharmacokinetics for DRF

To show that an engineered human CGL enzyme that degrades homocysteine and homocystine could lower toxic levels of tHcy in plasma, in vivo PD and efficacy studies were conducted with a polyhistidine tagged and/or PEGylated version of enzyme molecule CGL-ILMDRGVS (e.g., HIS-CGL-ILMDRGVS-PEG or CGL-ILMDRGVS-PEG). Additionally, CGL-ILMDRGVS-PEG was used for confirmatory PK/PD studies in cynomolgus monkeys, and PD, while HIS-CGL-ILMDRGVS-PEG was used for efficacy studies in the CBS-deficient (CBS−/−) mouse model.

CBS−/− Mouse Model

The CBS-deficient (CBS−/−) mouse model is a clinically relevant mouse model of classical homocystinuria (homocystinuria due to CBS deficiency). Elevated plasma tHcy levels and the downstream disease manifestations were evaluated using the CBS−/− mouse model. The CBS−/− mouse model is a clinically relevant mouse model of classical homocystinuria (homocystinuria due to CBS deficiency) that possesses many of the characteristics of the human disease, including hyperhomocysteinemia, osteoporosis, and developmental deficits. Homozygous CBS−/− mice suffer from neonatal lethality due to their genetic defect, but life expectancy can be increased with access to betaine, which remethylates homocysteine to methionine, at an early age. However, betaine only improves survival in 50% of CBS−/− mice when given access via mother's milk through to the age of weaning, indicating a need for a more effective therapy.

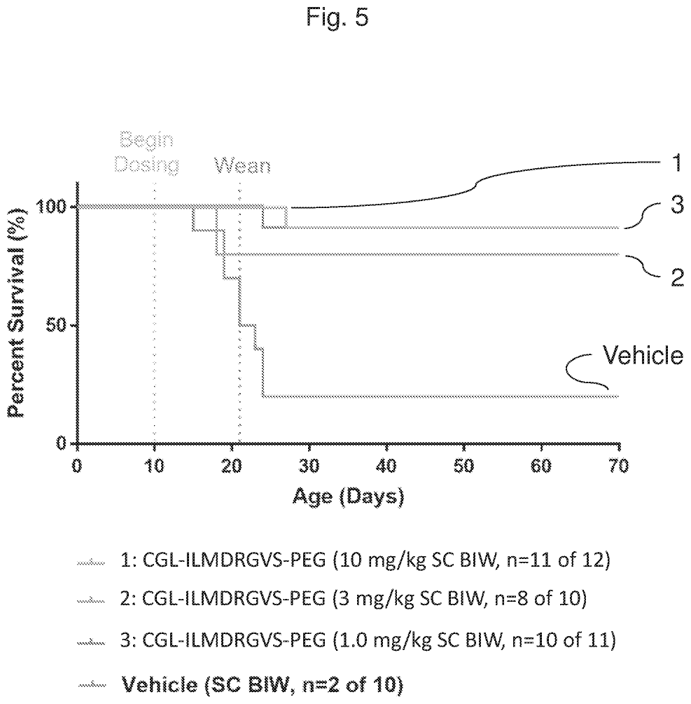

The CBS−/− mice in the study were treated with dose levels ranging from 3.125 to 25 mg/kg of HIS-CGL-ILMDRGVS-PEG with subcutaneous (SC) dosing twice per week (BIW) to determine if decreasing plasma tHcy levels in the CBS−/− mouse improves survival. Additionally, intraperitoneal (IP) dosing was tested at 25 mg/kg BIW and SC dosing once per week (QW) at 12.5 mg/kg. In the study, all animals had access to betaine via mother's milk from birth until weaning to enhance survival. Dosing with HIS-CGL-ILMDRGVS-PEG began at day 10 of age and continued until the animals were at least 12 weeks old when animals were fully mature and no longer were observed to be in danger of impaired survival. Statistically significant improvements in survival relative to PBS control (˜15% survival) have been observed for all dose levels down to 3.125 mg/kg SC where no animal deaths were reported. In addition, clear improvements in liver pathology were observed (FIG. 5, 6) indicating that HIS-CGL-ILMDRGVS-PEG is a superior therapy to betaine in animals with CBS deficiency. The observed survival effect of HIS-CGL-ILMDRGVS-PEG in the CBS−/− mice was used to undertake the preliminary PD analyses.

Pharmacokinetic and Pharmacodynamic Studies in Monkeys

Intravenous or subcutaneous doses of CGL-ILMDRGVS-PEG were administered to male cynomolgus monkeys. The study characterized the PK and PD of CGL-ILMDRGVS-PEG after IV or SC dosing at 2 and 8 mg/kg. Evaluation of site of injection following weekly SC administration for 3 weeks was performed in phase 2 of the study.

Total (free and bound) homocysteine and its oxidized form homocystine, (referred to as total homocysteine (tHCY) was used as a pharmacodynamic marker. Mean clearance (CL) appeared slightly lower at the lower dose but the ranges (mean±SD) were overlapping after IV administration. The observed volume of distribution (Vss) was mostly comparable to monkey serum volume (45 mL/kg) although the mean estimates trended a bit lower. The resulting mean IV half-life (T½) estimates ranged from 88.4 to 93.6 hours (˜3.7-3.9 days). Increases in IV exposure (Cₘₐₓ, AUC_{0-∞}) were close to dose proportional and there were ˜3.6-fold and ˜3.4-fold increases in mean Cₘₐₓ and AUC_{0-∞} for a 4-fold change in dose. Median Tₘₐₓ was observed at 0.5 hours post dose.

SC exposure (Cₘₐₓ, AUC_{0-∞}) was higher at 8 vs. 2 mg/kg. Median Tₘₐₓ was later for the 8 mg/kg dose at 48 hours versus 24 hours at 2 mg/kg. The mean bioavailability estimate at 8 mg/kg was moderate (75.8%). Bioavailability was not evaluated at the 2 mg/kg dose.

Following IV administration of CGL-ILMDRGVS-PEG, maximal reduction of plasma tHCY levels were observed at 4-8 hours post dose and additional mean reduction was observed for the higher IV dose level of 8 mg/kg. There was an initial ˜39% and ˜72% drop in plasma tHCY levels at 5 min post dose (at 2 and 8 mg/kg respectively), followed by a slower decline to the maximal observed suppression. At 8 hours post dose, mean tHCY levels were at 47.8% and 18.1% of the average baseline levels (corresponding to 52.2% and 81.9% decreases), respectively. This corresponded to mean CGL-ILMDRGVS-PEG concentrations at or above 58.6 and 194 μg/mL, respectively. Recovery to baseline tHCY levels was mostly complete by 240 hours for the 2 mg/kg IV dose group and ˜84% complete by 336 hours (2 weeks) for the 8 mg/kg IV dose group.

Following SC administration of CGL-ILMDRGVS-PEG, maximal reduction of plasma tHCY levels was observed at 24-48 hours post dose at the 8 mg/kg dose level and at 8-24 hours for the 2 mg/kg dose. There was a steady decline in tHCY levels until maximal reduction was achieved. At the time of maximal reduction, mean tHCY levels were at 75.5% and 42.0% of the average baseline levels (corresponding to 24.5% and 58.0% decreases) for the 2 and 8 mg/kg dose levels, respectively. This corresponded to mean CGL-ILMDRGVS-PEG concentrations up to 34.9 and 98.7 μg/mL, respectively, noting that there was marked inter-animal variability at 2 mg/kg. Recovery to baseline tHCY levels was complete by 336 hours or Day 15 predose (2 weeks) for the 8 mg/kg/Day 1 dose. Following the 2 mg/kg dose on Day 15, the recovery appeared complete by 168 hours post dose.

Dosing and Administration

Accordingly, one or more embodiments of the present invention provide a dosing regimen for a modified CGL enzyme such as CGL-ILMDRGVS or CGL-ILMDRGVS-PEG. In various embodiments, the enzyme is administered at a dose of about 0.05 mg/kg to about 4 mg/kg, such as about 0.05 mg/kg, about 0.1 mg/kg, about 0.15 mg/kg, about 0.2 mg/kg, about 0.25 mg/kg, about 0.3 mg/kg, about 0.35 mg/kg, about 0.4 mg/kg, about 0.45 mg/kg, about 0.5 mg/kg, about 0.55 mg/kg, about 0.6 mg/kg, about 0.65 mg/kg, about 0.7 mg/kg, about 0.75 mg/kg, about 0.8 mg/kg, about 0.85 mg/kg, about 0.9 mg/kg, about 0.95 mg/kg, about 1 mg/kg, about 1.1 mg/kg, about 1.2 mg/kg, about 1.3 mg/kg, about 1.4 mg/kg, about 1.5 mg/kg, about 2 mg/kg, about 2.5 mg/kg, about 3 mg/kg, about 3.5 mg/kg or about 4 mg/kg. For PEGylated enzymes (e.g. CGL-ILMDRGVS-PEG), the enzyme dose is calculated based on the unPEGylated form of the enzyme (e.g. CGL-ILMDRGVS).

In one or more embodiments, the modified CGL enzyme (e.g. CGL-ILMDRGVS or CGL-ILMDRGVS-PEG) (or composition comprising such enzyme) is administered in multiple doses. In various embodiments, the enzyme is administered once a day to once a month, such as once a day, once every two days, once every three days, once every four days, once every five days, once every six days, once a week, once every other week, once every third week or once a month.

Exemplary dosing regimens include about 0.05 mg/kg to about 4 mg/kg administered at a frequency of once a day to once a month. In one or more embodiments, the enzyme is administered at a dose of 0.1 mg/kg at a frequency of once a week. In one or more embodiments, the enzyme is administered at a dose of 0.15 mg/kg at a frequency of once a week. In one or more embodiments, the enzyme is administered at a dose of 0.2 mg/kg at a frequency of once a week. In one or more embodiments, the enzyme is administered at a dose of 0.25 mg/kg at a frequency of once a week. In one or more embodiments, the enzyme is administered at a dose of 0.3 mg/kg at a frequency of once a week. In one or more embodiments, the enzyme is administered at a dose of 0.35 mg/kg at a frequency of once a week. In one or more embodiments, the enzyme is administered at a dose of 0.4 mg/kg at a frequency of once a week. In one or more embodiments, the enzyme is administered at a dose of 0.45 mg/kg at a frequency of once a week. In one or more embodiments, the enzyme is administered at a dose of 0.5 mg/kg at a frequency of once a week. In one or more embodiments, the enzyme is administered at a dose of 0.6 mg/kg at a frequency of once a week. In one or more embodiments, the enzyme is administered at a dose of 0.7 mg/kg at a frequency of once a week. In one or more embodiments, the enzyme is administered at a dose of 0.8 mg/kg at a frequency of once a week. In one or more embodiments, the enzyme is administered at a dose of 0.9 mg/kg at a frequency of once a week. In one or more embodiments, the enzyme is administered at a dose of 1 mg/kg at a frequency of once a week. In one or more embodiments, the enzyme is administered at a dose of 1.5 mg/kg at a frequency of once a week. In one or more embodiments, the enzyme is administered at a dose of 2 mg/kg at a frequency of once a week. In one or more embodiments, the enzyme is administered at a dose of 2.5 mg/kg at a frequency of once a week. In one or more embodiments, the enzyme is administered at a dose of 3 mg/kg at a frequency of once a week. In one or more embodiments, the enzyme is administered at a dose of 3.5 mg/kg at a frequency of once a week. In one or more embodiments, the enzyme is administered at a dose of 4 mg/kg at a frequency of once a week.

The modified CGL enzyme as described herein (or composition comprising such enzyme) can be administered via any appropriate route, including intravenously, intrathecally, subcutaneously, intramuscularly, intratumorally, and/or intraperitoneally. In one or more embodiments, the modified CGL enzyme (or compositions comprising such enzyme) is administered intravenously (IV) or subcutaneously (SC).

Cystathionine-γ-Lyase

A lyase is an enzyme that catalyzes the breaking of various chemical bonds, often forming a new double bond or a new ring structure. For example, an enzyme that catalyzes this reaction would be a lyase: ATP-----+cAMP+PPi. Lyases differ from other enzymes in that they only require one substrate for the reaction in one direction, but two substrates for the reverse reaction.

A number of pyrioxal-5′-phosphate (PLP)-dependent enzymes are involved in the metabolism of cysteine, homocysteine, and methionine, and these enzymes form an evolutionarily related family, designated as Cys/Met metabolism PLP-dependent enzymes. These enzymes are proteins of about 400 amino acids and the PLP group is attached to a lysine residue located in the central location of the polypeptide. Members of this family include cystathionine-γ-lyase (CGL), cystathionine-γ-synthase (CGS), cystathionine-β-lyase (CBL), methionine-γ-lyase (MGL), and O-acetylhomoserine (OAH)/O-acetyl-serine (OAS) sulfhydrylase (OSHS). Common to all of them is the formation of a Michaelis complex leading to an external substrate aldimine. The further course of the reaction is determined by the substrate specificity of the particular enzyme.

For example, specific mutations were introduced into a PLP-dependent lyase family member, such as the human cystathionine-γ-lyase, to change its substrate specificity. In this manner novel variants were produced with the de nova ability to degrade homocyst(e)ine as a substrate with higher catalytic activity than hGGL-NLV. A modification of other PLP-dependent enzymes for producing novel homocyst(e)ine degrading activity may also be contemplated.

CGL is a tetramer that catalyzes the last step in the mammalian transsulfuration pathway (Rao et al., 1990). CGL catalyzes the conversion of L-cystathionine to L-cysteine, alpha-ketobutyrate, and ammonia. Pyridoxal phosphate is a prosthetic group of this enzyme. Protein engineering was used to convert cystathionase, which has only weak activity for the degradation of homocysteine and homocystine, into an enzyme that can degrade homocysteine and homocystine at a high rate (U.S. Pat. No. 9,481,877, which is incorporated herein by reference in its entirety).

Homocyst(e)inase Engineering

Since humans do not produce homocyst(e)inase it is necessary to engineer homocyst(e)inase for human therapy that have high activity and specificity for degrading homocyst(e)ine under physiological conditions, as well as high stability in physiological fluids such as serum, and are also non-immunogenic because they are native proteins that normally elicit immunological tolerance.

Due to the undesired immunogenic effects seen in animal studies with pMGL (MGL from P. putida), it is desirable to engineer homocyst(e)ine degradation activity in a human enzyme. Immunological tolerance to human proteins makes it likely that such an enzyme will be non-immunogenic or minimally immunogenic and therefore well tolerated.

Although mammals do not have a homocyst(e)inase, they do have a cystathionine-γ-lyase (CGL). CGL is a tetramer that catalyzes the last step in the mammalian transsulfuration pathway (Rao et al., 1990). CGL catalyzes the conversion of L-cystathionine to L-cysteine, alpha-ketobutyrate, and ammonia. The human CGL (hCGL) cDNA had previously been cloned and expressed, but with relatively low yields (˜5 mg/L culture) (Lu et al., 1992; Steegbom et al., 1999).

As such, there are provided methods and compositions related to a primate (particularly human) cystathionine-γ-lyase (CGL or cystathionase) modified via mutagenesis to hydrolyze homocyst(e)ine with high efficiency.

Described are modified CGL enzymes that exhibit at least one functional activity that is comparable to an unmodified CGL enzyme. A modified CGL enzyme may be further modified to increase serum stability. Modified CGL enzymes include, for example, a protein that possesses an additional advantage, such as the homocyst(e)inase enzyme activity, compared to the unmodified CGL enzyme. The unmodified protein or polypeptide may be a native cystathionine-γ-lyase, such as a human cystathionine-γ-lyase.

Determination of activity may be achieved using assays familiar to those of skill in the art, particularly with respect to the enzyme's activity, and may include for comparison purposes, for example, the use of native and/or recombinant versions of either the modified or unmodified enzyme. For example, the homocyst(e)inase activity may be determined by any assay to detect the production of any substrates resulting from conversion of homocyst(e)ine, such as alpha-ketobutyrate, methanethiol, and/or ammonia.

A modified CGL enzyme, may be identified based on its increase in homocyst(e)ine degrading activity. For example, substrate recognition sites of the unmodified polypeptide may be identified. This identification may be based on structural analysis or homology analysis. A population of mutants involving modifications of such substrate recognitions sites may be generated. Mutants with increased homocyst(e)ine degrading activity may be selected from the mutant population. Selection of desired mutants may include methods, such as detection of byproducts or products from homocyst(e)ine degradation.

Modified CGL enzymes may possess deletions and/or substitutions of amino acids; thus, an enzyme with a deletion, an enzyme with a substitution, and an enzyme with a deletion and a substitution are modified CGL enzymes. These modified CGL enzymes may further include insertions or added amino acids, such as with fusion proteins or proteins with linkers, for example. A “modified deleted CGL enzyme” lacks one or more residues of the native enzyme, but may possess the specificity and/or activity of the native enzyme. A modified deleted CGL enzyme may also have reduced immunogenicity or antigenicity. An example of a modified deleted CGL enzyme is one that has an amino acid residue deleted from at least one antigenic region, that is, a region of the enzyme determined to be antigenic in a particular organism, such as the type of organism that may be administered the modified CGL enzyme.

Substitution or replacement variants may contain the exchange of one amino acid for another at one or more sites within the protein and may be designed to modulate one or more properties of the polypeptide, particularly its effector functions and/or bioavailability. Substitutions may or may not be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine, or methionine; serine to threonine; threonine to serine; tryptophan to tyrosine; tyrosine to tryptophan or phenylalanine; and valine to isoleucine or leucine.

In addition to a deletion or substitution, a modified CGL enzyme may possess an insertion of residues, which typically involves the addition of at least one residue in the enzyme. This may include the insertion of a targeting peptide or polypeptide or simply a single residue. Terminal additions, called fusion proteins, are discussed below.

The term “biologically functional equivalent” is well understood in the art and is further defined in detail herein. Accordingly, CGL enzyme sequences that have about 90% or more sequence identity to SEQ ID NO: 1, or even between about 91% and about 99% of amino acids (including 92%, 93%, 94%, 95%, 96%, 97%, 98%, and 99%) that are identical to or conservative substitution of the amino acids of an modified CGL enzyme disclosed herein 10 are included, provided the biological activity of the enzyme is maintained such that a 15 measureable biological activity parameter (e.g., conversion of homocyst(e)ine to alphaketobutyrate, methanethiol, and ammonia) is within about 20%, about 15%, about 10%, or about 5% of a modified CGL enzyme disclosed herein. A modified CGL enzyme may be biologically functionally equivalent to its unmodified counterpart.

It also will be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various noncoding sequences flanking either of the 5′ or 3′ portions of the coding region or may include various internal sequences, i.e., introns, which are known to occur within genes.

Conjugates

The compositions and methods provided involve further modification of the modified CGL enzyme for improvement, such as by forming conjugates with heterologous peptide segments or polymers, such as polyethylene glycol. The modified CGL enzyme may be linked to PEG to increase the hydrodynamic radius of the enzyme and hence increase the serum persistence. The disclosed polypeptide may be conjugated to any targeting agent, such as a ligand having the ability to specifically and stably bind to an external receptor or binding site on a tumor cell (U.S. Patent Publ. 2009/0304666). The PEG can be from about 3,000 to 20,000 Daltons in size, with an exemplary size being about 5,000 Daltons.

Fusion Proteins

Fusion proteins are provided in which the modified CGL enzyme may be linked at the N- or C-terminus to a heterologous domain. For example, fusions may also employ leader sequences from other species to permit the recombinant expression of a protein in a heterologous host. Another useful fusion includes the addition of a protein affinity tag, such as a serum albumin affinity tag or six histidine residues, or an immunologically active domain, such as an antibody epitope, preferably cleavable, to facilitate purification of the fusion protein. Non-limiting affinity tags include polyhistidine, chitin binding protein (CBP), maltose binding protein (MBP), and glutathione-S-transferase (GST).

The modified CGL enzyme may be linked to a peptide that increases the in vivo half-life, such as an XTEN polypeptide (Schellenberger et al., 2009), IgG Fe domain, albumin, or an albumin binding peptide.

Methods of generating fusion proteins are well known to those of skill in the art. Such proteins can be produced, for example, by de nova synthesis of the complete fusion protein, or by attachment of the DNA sequence encoding the heterologous domain, followed by expression of the intact fusion protein.

Production of fusion proteins that recover the functional activities of the parent proteins may be facilitated by connecting genes with a bridging DNA segment encoding a peptide linker that is spliced between the polypeptides connected in tandem. The linker would be of sufficient length to allow proper folding of the resulting fusion protein.

Linkers

The modified CGL enzyme may be chemically conjugated using bifunctional cross-linking reagents or fused at the protein level with peptide linkers. Bifunctional crosslinking reagents have been extensively used for a variety of purposes, including preparation of affinity matrices, modification and stabilization of diverse structures, identification of ligand and receptor binding sites, and structural studies. Suitable peptide linkers may also be used to link the modified CGL enzyme, such as Gly-Ser linkers.

Homobifunctional reagents that carry two identical functional groups may induce cross-linking between identical and different macromolecules or subunits of a macromolecule, and link polypeptide ligands to their specific binding sites. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino-, sulfhydryl, guanidine-, indole-, carboxyl-specific groups. Of these, reagents directed to free amino groups have become popular because of their commercial availability, ease of synthesis, and the mild reaction conditions under which they can be applied.

Some heterobifunctional cross-linking reagents contain a primary aminereactive group and a thiol-reactive group. In another example, heterobifunctional cross-linking reagents and methods of using the cross-linking reagents are described (U.S. Pat. No. 5,889,155, specifically incorporated herein by reference in its entirety). The cross-linking reagents combine a nucleophilic hydrazide residue with an electrophilic maleimide residue, allowing coupling, in one example, of aldehydes to free thiols. The cross-linking reagent can be modified to cross-link various functional groups.

Additionally, any other linking/coupling agents and/or mechanisms known to those of skill in the art may be used to combine modified CGL enzymes, such as, for example, antibody-antigen interaction, avidin biotin linkages, amide linkages, ester linkages, thioester linkages, ether linkages, thioether linkages, phosphoester linkages, phosphoramide linkages, anhydride linkages, disulfide linkages, ionic and hydrophobic interactions, bispecific antibodies and antibody fragments, or combinations thereof.

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo. These linkers are thus one group of linking agents.

In addition to hindered cross-linkers, non-hindered linkers also can be employed in accordance herewith. Other useful cross-linkers, not considered to contain or generate a protected disulfide, include SATA, SPDP, and 2-iminothiolane (Wawrzynczak and Thorpe, 1987). The use of such cross-linkers is well understood in the art. Flexible linkers may also be used.

Once chemically conjugated, the peptide generally will be purified to separate the conjugate from unconjugated agents and from other contaminants. A large number of purification techniques are available for use in providing conjugates of a sufficient degree of purity to render them clinically useful.

Purification methods based upon size separation, such as gel filtration, gel permeation, or high performance liquid chromatography, will generally be of most use. Other chromatographic techniques, such as Blue-Sepharose separation, may also be used. Conventional methods to purify the fusion proteins from inclusion bodies may be useful, such as using weak detergents, such as sodium N-lauroyl-sarcosine (SLS).

PEGylation

Methods and compositions related to PEGylation of modified CGL enzyme are disclosed. For example, the modified CGL enzyme may be PEGylated in accordance with the methods disclosed herein.

PEGylation is the process of covalent attachment of poly(ethylene glycol) polymer chains to another molecule, normally a drug or therapeutic protein. PEGylation is routinely achieved by incubation of a reactive derivative of PEG with the target macromolecule. The covalent attachment of PEG to a drug or therapeutic protein can “mask” the agent from the host's immune system (reduced immunogenicity and antigenicity) or increase the hydrodynamic size (size in solution) of the agent, which prolongs its circulatory time by reducing renal clearance. PEGylation can also provide water solubility to hydrophobic drugs and proteins.

The first step of the PEGylation is the suitable functionalization of the PEG polymer at one or both terminals. PEGs that are activated at each terminus with the same reactive moiety are known as “homobifunctional,” whereas if the functional groups present are different, then the PEG derivative is referred as “heterobifunctional” or “heterofunctional.” The chemically active or activated derivatives of the PEG polymer are prepared to attach the PEG to the desired molecule.

The choice of the suitable functional group for the PEG derivative is based on the type of available reactive group on the molecule that will be coupled to the PEG. For proteins, typical reactive amino acids include lysine, cysteine, histidine, arginine, aspartic acid, glutamic acid, serine, threonine, and tyrosine. The N-terminal amino group and the C-terminal carboxylic acid can also be used.

The techniques used to form first generation PEG derivatives are generally reacting the PEG polymer with a group that is reactive with hydroxyl groups, typically anhydrides, acid chlorides, chloroformates, and carbonates. In the second generation PEGylation chemistry more efficient functional groups, such as aldehyde, esters, amides, etc., are made available for conjugation.

As applications of PEGylation have become more and more advanced and sophisticated, there has been an increase in need for heterobifunctional PEGs for conjugation. These heterobifunctional PEGs are very useful in linking two entities, where a hydrophilic, flexible, and biocompatible spacer is needed. Preferred end groups for heterobifunctional PEGs are maleimide, vinyl sulfones, pyridyl disulfide, amine, carboxylic acids, and NHS esters.

The most common modification agents, or linkers, are based on methoxy PEG (mPEG) molecules. Their activity depends on adding a protein-modifying group to the alcohol end. In some instances polyethylene glycol (PEG diol) is used as the precursor molecule. The diol is subsequently modified at both ends in order to make a hetero- or homo-dimeric PEGlinked molecule.

Proteins are generally PEGylated at nucleophilic sites, such as unprotonated thiols (cysteinyl residues) or amino groups. Examples of cysteinyl-specific modification reagents include PEG maleimide, PEG iodoacetate, PEG thiols, and PEG vinylsulfone. All four are strongly cysteinyl-specific under mild conditions and neutral to slightly alkaline pH but each has some drawbacks. The thioether formed with the maleimides can be somewhat unstable under alkaline conditions so there may be some limitation to formulation options with this linker. The carbamothioate linkage formed with iodo PEGs is more stable, but free iodine can modify tyrosine residues under some conditions. PEG thiols form disulfide bonds with protein thiols, but this linkage can also be unstable under alkaline conditions. PEG-vinylsulfone reactivity is relatively slow compared to maleimide and iodo PEG; however, the thioether linkage formed is quite stable. Its slower reaction rate also can make the PEG-vinylsulfone reaction easier to control.

Site-specific PEGylation at native cysteinyl residues is seldom carried out, since these residues are usually in the form of disulfide bonds or are required for biological activity. On the other hand, site-directed mutagenesis can be used to incorporate cysteinyl PEGylation sites for thiol-specific linkers. The cysteine mutation must be designed such that it is accessible to the PEGylation reagent and is still biologically active after PEGylation.

Amine-specific modification agents include PEG NHS ester, PEG tresylate, PEG aldehyde, PEG isothiocyanate, and several others. All react under mild conditions and are very specific for amino groups. The PEG NHS ester is probably one of the more reactive agents; however, its high reactivity can make the PEGylation reaction difficult to control on a large scale. PEG aldehyde forms an imine with the amino group, which is then reduced to a secondary amine with sodium cyanoborohydride. Unlike sodium borohydride, sodium cyanoborohydride will not reduce disulfide bonds. However, this chemical is highly toxic and must be handled cautiously, particularly at lower pH where it becomes volatile.

Due to the multiple lysine residues on most proteins, site-specific PEGylation can be a challenge. Fortunately, because these reagents react with unprotonated amino groups, it is possible to direct the PEGylation to lower-pK amino groups by performing the reaction at a lower pH. Generally the pK of the alpha-amino group is 1-2 pH units lower than the epsilon-amino group of lysine residues. By PEGylating the molecule at pH 7 or below, high selectivity for the N-terminus frequently can be attained. However, this is only feasible if the N-terminal portion of the protein is not required for biological activity. Still, the pharmacokinetic benefits from PEGylation frequently outweigh a significant loss of in vitro bioactivity, resulting in a product with much greater in vivo bioactivity regardless of PEGylation chemistry.

There are several parameters to consider when developing a PEGylation procedure. Fortunately, there are usually no more than four or five parameters. The “design of experiments” approach to optimization of PEGylation conditions can be very useful. For thiol-specific PEGylation reactions, parameters to consider include: protein concentration, PEG-to-protein ratio (on a molar basis), temperature, pH, reaction time, and in some instances, the exclusion of oxygen. (Oxygen can contribute to intermolecular disulfide formation by the protein, which will reduce the yield of the PEGylated product.) The same factors should be considered (with the exception of oxygen) for amine-specific modification except that pH may be even more critical, particularly when targeting the N-terminal amino group.

For both amine- and thiol-specific modifications, the reaction conditions may affect the stability of the protein. This may limit the temperature, protein concentration, and pH. In addition, the reactivity of the PEG linker should be known before starting the PEGylation reaction. For example, if the PEGylation agent is only 70% active, the amount of PEG used should ensure that only active PEG molecules are counted in the protein-to-PEG reaction stoichiometry.

Proteins and Peptides

Compositions comprising at least one protein or peptide, such as a modified CGL enzyme, are provided. These peptides may be comprised in a fusion protein or conjugated to an agent as described supra.

As used herein, a protein or peptide generally refers, but is not limited to, a protein of greater than about 200 amino acids, up to a full-length sequence translated from a gene; a polypeptide of greater than about 100 amino acids; and/or a peptide of from about 3 to about 100 amino acids. For convenience, the terms “protein,” “polypeptide,” and “peptide” are used interchangeably herein.

Accordingly, the term “protein or peptide” encompasses amino acid sequences comprising at least one of the 20 common amino acids found in naturally occurring proteins, or at least one modified or non-natural amino acid.

Proteins or peptides may be made by any technique known to those of skill in the art, including the expression of proteins, polypeptides, or peptides through standard molecular biological techniques, the isolation of proteins or peptides from natural sources, or the chemical synthesis of proteins or peptides. The coding regions for known genes may be amplified and/or expressed using the techniques disclosed herein or as would be known to those of ordinary skill in the art. Alternatively, various commercial preparations of proteins, polypeptides, and peptides are known to those of skill in the art.

Nucleic Acids and Vectors

Nucleic acid sequences encoding a modified CGL enzyme or a fusion protein containing a modified CGL enzyme are disclosed. Depending on which expression system is used, nucleic acid sequences can be selected based on conventional methods. For example, if the modified CGL enzyme is derived from human cystathionase and contains multiple codons that are rarely utilized in E. coli, then that may interfere with expression. Therefore, the respective genes or variants thereof may be codon optimized for E. coli expression using freely available software (see Hoover & Lubkowski, 2002) to design coding sequences free of rare codons. Various vectors may be also used to express the protein of interest, such as a modified CGL enzyme. Exemplary vectors include, but are not limited, plasmid vectors, viral vectors, transposon, or liposome-based vectors.

Host Cells

Host cells may be any that may be transformed to allow the expression and secretion of modified CGL enzyme and conjugates thereof. The host cells may be bacteria, mammalian cells, yeast, or filamentous fungi. Various bacteria include Escherichia and Bacillus. Yeasts belonging to the genera Saccharomyces, Kiuyveromyces, Hansenula, or Pichia would find use as an appropriate host cell. Various species of filamentous fungi may be used as expression hosts, including the following genera: Aspergillus, Trichoderma, Neurospora, Penicillium, Cephalosporium, Achlya, Podospora, Endothia, Mucor, Cochliobolus, and Pyricularia.

Examples of usable host organisms include bacteria, e.g., Escherichia coli MC1061, derivatives of Bacillus subtilis BRB1 (Sibakov et al., 1984), Staphylococcus aureus SAI123 (Lordanescu, 1975) or Streptococcus lividans (Hopwood et al., 1985); yeasts, e.g., Saccharomyces cerevisiae AH 22 (Mellor et al., 1983) or Schizosaccharomyces pombe; and filamentous fungi, e.g., Aspergillus nidulans, Aspergillus awamori (Ward, 1989), or Trichoderma reesei (Penttila et al., 1987; Harkki et al., 1989).

Examples of mammalian host cells include Chinese hamster ovary cells (CHO-Kl; American Type Culture Collection (ATCC) No. CCL61), rat pituitary cells (GHl; ATCC No. CCL82), human cervix cells (HeLa S3; ATCC No. CCL2.2), rat hepatoma cells (H-4-11-E; ATCC No. CRL-1548), SV40-transformed monkey kidney cells (COS-1; ATCC No. CRL-1650), and murine embryonic fibroblast cells (NIH-3T3; ATCC No. CRL-1658). The foregoing is meant to be illustrative but not limitative of the many possible host organisms known in the art. In principle, all hosts capable of secretion can be used whether prokaryotic or eukaryotic.

Mammalian host cells expressing the modified CGL enzymes and/or their fusion proteins are cultured under conditions typically employed to culture the parental cell line. Generally, cells are cultured in a standard medium containing physiological salts and nutrients, such as standard RPMI, MEM, IMEM, or DMEM, typically supplemented with 5%-10% serum, such as fetal bovine serum. Culture conditions are also standard, e.g., cultures are incubated at 37° C. in stationary or roller cultures until desired levels of the proteins are achieved.

Protein Purification

Protein purification techniques are well known to those of skill in the art. These techniques involve, at one level, the homogenization and crude fractionation of the cells, tissue, or organ to polypeptide and non-polypeptide fractions. The protein or polypeptide of interest may be further purified using chromatographic and electrophoretic techniques to achieve partial or complete purification (or purification to homogeneity) unless otherwise specified. Analytical methods particularly suited to the preparation of a pure peptide are ion-exchange chromatography, gel exclusion chromatography, polyacrylamide gel electrophoresis, affinity chromatography, immunoaffinity chromatography, and isoelectric focusing. A particularly efficient method of purifying peptides is fast-performance liquid chromatography (FPLC) or even high-performance liquid chromatography (HPLC).

A purified protein or peptide is intended to refer to a composition, isolatable from other components, wherein the protein or peptide is purified to any degree relative to its naturally-obtainable state. An isolated or purified protein or peptide, therefore, also refers to a protein or peptide free from the environment in which it may naturally occur. Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95%, or more of the proteins in the composition.

Various techniques suitable for use in protein purification are well known to those of skill in the art. These include, for example, precipitation with ammonium sulfate, PEG, antibodies and the like, or by heat denaturation, followed by centrifugation; chromatography steps, such as ion exchange, gel filtration, reverse phase, hydroxyapatite, and affinity chromatography; isoelectric focusing; gel electrophoresis; and combinations of these and other techniques. As is generally known in the art, it is believed that the order of conducting the various purification steps may be changed, or that certain steps may be omitted, and still result in a suitable method for the preparation of a substantially purified protein or peptide.

Various methods for quantifying the degree of purification of the protein or peptide are known to those of skill in the art. These include, for example, determining the specific activity of an active fraction, or assessing the amount of polypeptides within a fraction by sodium dodecyl sulfate polyacrylamide gel electrophoresis (SDS/PAGE) analysis. A preferred method for assessing the purity of a fraction is to calculate the specific activity of the fraction, to compare it to the specific activity of the initial extract, and to thus calculate the degree of purity therein, assessed by a “fold purification number.” The actual units used to represent the amount of activity will, of course, be dependent upon the particular assay technique chosen to follow the purification, and whether or not the expressed protein or peptide exhibits a detectable activity.

There is no general requirement that the protein or peptide will always be provided in its most purified state. Indeed, it is contemplated that less substantially purified products may have utility. Partial purification may be accomplished by using fewer purification steps in combination, or by utilizing different forms of the same general purification scheme. For example, it is appreciated that a cation-exchange column chromatography performed utilizing a high-performance liquid chromatography (HPLC) apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

A protein or peptide may be isolated or purified, for example, a modified CGL enzyme, a fusion protein containing the modified CGL enzyme, or a modified CGL enzyme post PEGylation. For example, a His tag or an affinity epitope may be comprised in such a modified CGL enzyme to facilitate purification. Affinity chromatography is a chromatographic procedure that relies on the specific affinity between a substance to be isolated and a molecule to which it can specifically bind. This is a receptor-ligand type of interaction. The column material is synthesized by covalently coupling one of the binding partners to an insoluble matrix. The column material is then able to specifically adsorb the substance from the solution. Elution occurs by changing the conditions to those in which binding will not occur (e.g., altered pH, ionic strength, temperature, etc.). The matrix should be a substance that does not adsorb molecules to any significant extent and that has a broad range of chemical, physical, and thermal stability. The ligand should be coupled in such a way as to not affect its binding properties. The ligand should also provide relatively tight binding. It should be possible to elute the substance without destroying the sample or the ligand.

Size exclusion chromatography (SEC) is a chromatographic method in which molecules in solution are separated based on their size, or in more technical terms, their hydrodynamic volume. It is usually applied to large molecules or macromolecular complexes, such as proteins and industrial polymers. Typically, when an aqueous solution is used to transport the sample through the column, the technique is known as gel filtration chromatography, versus the name gel permeation chromatography, which is used when an organic solvent is used as a mobile phase.

The underlying principle of SEC is that particles of different sizes will elute (filter) through a stationary phase at different rates. This results in the separation of a solution of particles based on size. Provided that all the particles are loaded simultaneously or near simultaneously, particles of the same size should elute together. Each size exclusion column has a range of molecular weights that can be separated. The exclusion limit defines the molecular weight at the upper end of this range and is where molecules are too large to be trapped in the stationary phase. The permeation limit defines the molecular weight at the lower end of the range of separation and is where molecules of a small enough size can penetrate into the pores of the stationary phase completely and all molecules below this molecular mass are so small that they elute as a single band.

High-performance liquid chromatography (or high-pressure liquid chromatography, HPLC) is a form of column chromatography used frequently in biochemistry and analytical chemistry to separate, identify, and quantify compounds. HPLC utilizes a column that holds chromatographic packing material (stationary phase), a pump that moves the mobile phase(s) through the column, and a detector that shows the retention times of the molecules. Retention time varies depending on the interactions between the stationary phase, the molecules being analyzed, and the solvent(s) used.

Before describing several exemplary embodiments of the disclosure, it is to be understood that the disclosure is not limited to the details of construction or process steps set forth in the following description. The disclosure is capable of other embodiments and of being practiced or being carried out in various ways.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Rescue of CBS−/− Mice with Engineered Human Cystathionine γ-Lyase

CBS-deficient (CBS−/−) mice are used as an animal model for human homocystinuria due to CBS deficiency. The physiological presentation is similar to human disease in that mice present with elevated levels of plasma tHcy. Intraperitoneal (IP) and subcutaneous (SC) dosing of HIS-CGL-ILMDRGVS-PEG was assessed for a range of properties including the ability to enhance survival of CBS−/− mice. The mice were fed a normal (methionine-containing) diet, thus also providing a model for diet liberalization among human patients.

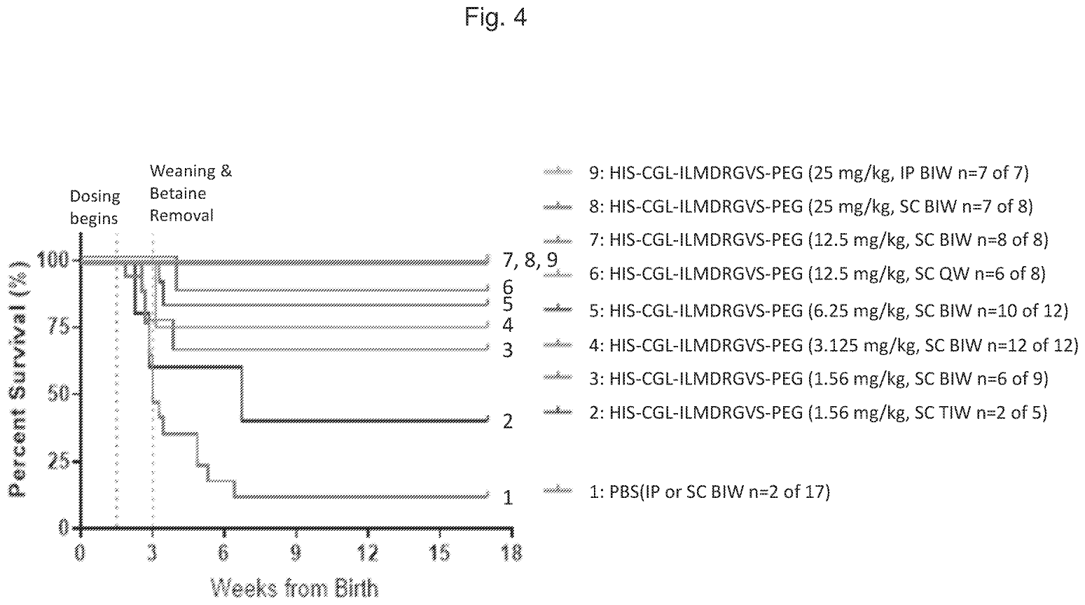

Mice were euthanized if physical well being deteriorated (e.g., due to morbidity), and this was recorded as survival time. CBS−/− mice were treated with doses ranging from 3.125 to 25 mg/kg of HIS-CGL-ILMDRGVS-PEG with SC dosing twice per week (BIW) to determine if decreasing plasma tHcy levels in the CBS−/− mouse improves survival (Table 3 and FIG. 4).

TABLE 3
CBS-/- mice Dosing Groups

| | Mice | Dose level | Dose | Dose | Dose Volume | |
|---|---|---|---|---|---|---|
| Dosing Material | (n=) | (mg/kg) | Route | Regimen | (mL/Kg) | Endpoint |
| PBS | 10 | N/A | IP | BIW | 3.26 | Survival |
| HIS-CGL- | 7 | 25.0 | IP | BIW | 3.26 | Survival |
| ILMDRGVS-PEG | | | | | | |
| HIS-CGL- | 8 | 25.0 | SC | BIW | 3.26 | Survival |
| ILMDRGVS-PEG | | | | | | |
| PBS | 7 | N/A | SC | BIW | 1.63 | Survival |
| HIS-CGL- | 8 | 12.5 | SC | BIW | 1.63 | Survival |
| ILMDRGVS-PEG | | | | | | |
| HIS-CGL- | 12 | 6.25 | SC | BIW | 1.63 | Survival |
| ILMDRGVS-PEG | | | | | | |
| HIS-CGL- | 12 | 3.125 | SC | BIW | 1.63 | Survival |
| ILMDRGVS-PEG | | | | | | |
| HIS-CGL- | 9 | 1.56 | SC | BIW | 1.63 | Survival |
| ILMDRGVS-PEG | | | | | | |
| HIS-CGL- | 8 | 12.5 | SC | QW | 1.63 | Survival |
| ILMDRGVS-PEG | | | | | | |
| HIS-CGL- | 5 | 1.56 | SC | TIW | 1.63 | Survival |
| ILMDRGVS-PEG | | | | | | |
| HIS-CGL- | 6 | 12.5 | SC | BIW | 1.63 | PD |
| ILMDRGVS-PEG | | | | | | |
| HIS-CGL- | 6 | 6.25 | SC | BIW | 1.63 | PD |
| ILMDRGVS-PEG | | | | | | |

Given that the human equivalent dose is approximately 1/12 of a mouse dose, the does in this study are human equivalent doses range from approximately 0.13 mg/kg to approximately 2 mg/kg.

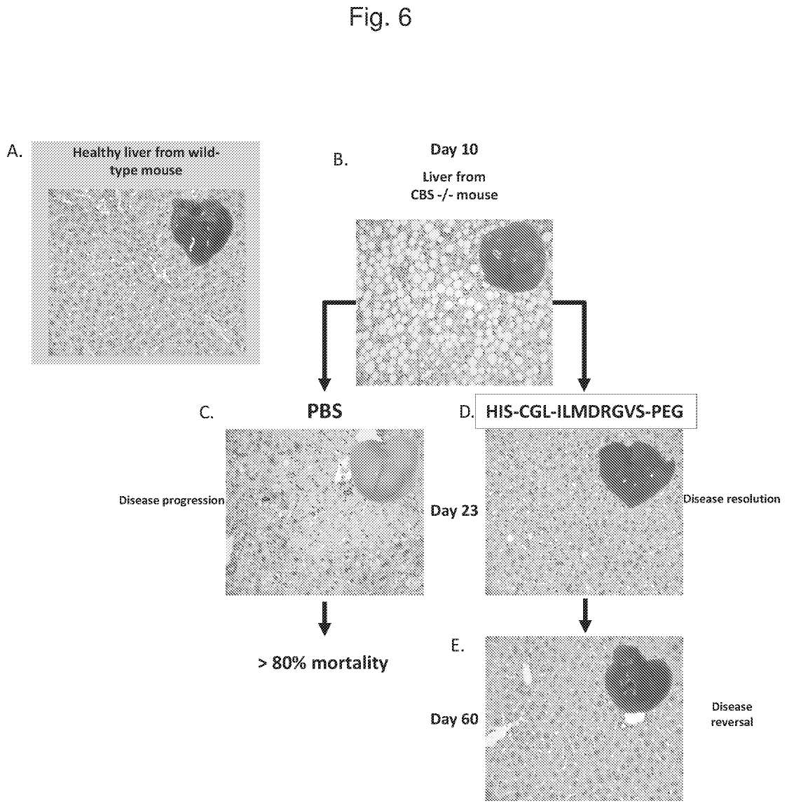

Depending on the study, newborn CBS−/− mice had access to betaine via mother's milk from birth until weaning to enhance survival and thus ensure adequate enrollment of animals into the experiment. Dosing with HTS-CGL-ILMDRGVS-PEG or CGL-ILMDRGVS-PEG began at day 10 of age and continued until the animals were at least 10 to 12 weeks old when animals were fully mature and no longer were observed to be in danger of impaired survival. Statistically significant improvements in survival relative to PBS control (˜15% survival) were observed for all dose levels down to 3.125 mg/kg SC where no animal deaths were reported (FIG. 4). In addition, clear improvements in liver pathology were observed (FIGS. 6-8) indicating that modified CGL enzymes (HIS-CGL-ILMDRGVS-PEG and CGL-ILMDRGVS-PEG) are a superior therapy to betaine in animals with CBS deficiency.

Through 16 weeks, dosing with 1.56 mg/kg HIS-CGL-ILMDRGVS-PEG TIW or PBS BIW resulted in 40% and 12% survival, respectively, while all tested dose levels of HIS-CGL-ILMDRGVS-PEG dosed BIW or QW resulted in greater than 50% survival (FIG. 4). There was 100% survival of mice dosed BIW with 12.5 or 3.125 mg/kg and 83% survival when dosed BIW with 6.25 mg/kg. Median survival was 3 weeks with PBS dosing and 6.75 weeks with TIW 1.56 mg/kg HIS-CGL-ILMDRGVS-PEG.

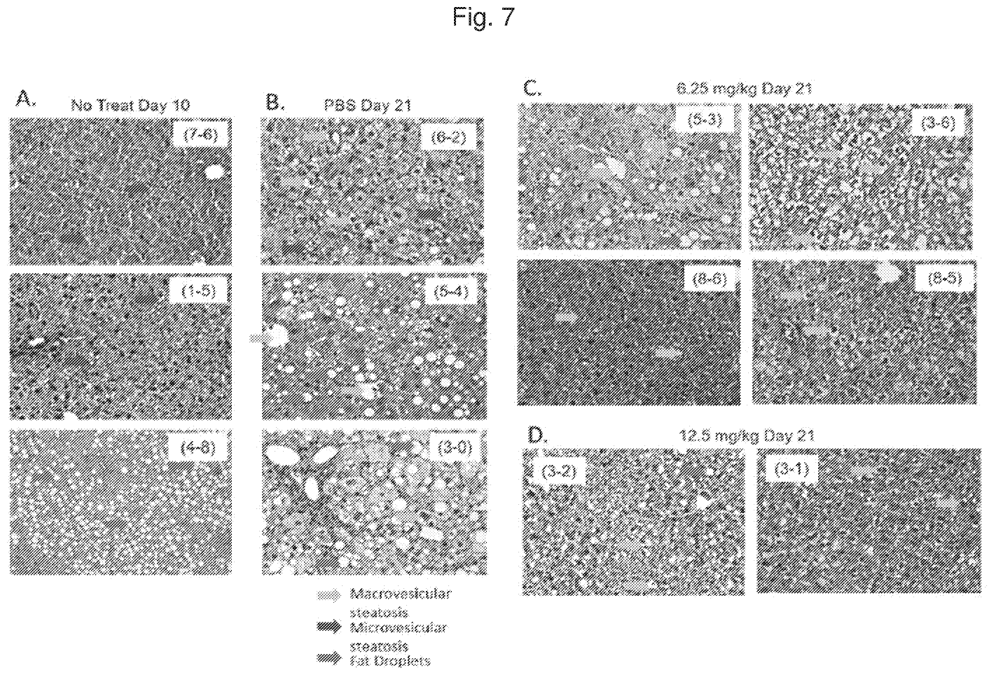

Pathology can be observed in the organs and tissues of CBS−/− mice as the disease state progresses. Liver samples were collected from healthy wild-type mice (FIG. 6A) or untreated CBS−/− mice and stained with Haemotoxylin and Eosin (H&E). In 10-day-old untreated mice, microvascular steatosis and the presence of fat droplets can be seen (FIG. 6B and FIG. 7A). Liver samples collected from 23-day old (FIG. 6C) and 21-day old (FIG. 7B) PBS treated mice and stained with H&E displayed increased microvascular steatosis, necrosis, fat droplets, and macrovascular steatosis compared to the samples from 10-day old untreated mice, as well as to samples from 23-day old (FIG. 6D) treated mice or 21-day old mice treated with either 6.25 or 12.5 mg/kg HIS-CGL-ILMDRGVS-PEG BIW (FIG. 7C, D). Liver samples collected from 60-day old mice treated with HIS-CGL-ILMDRGVS-PEG showed a disease reversal (FIG. 6E).

Liver samples were also stained with Oil-red-O. Oil-red-O stain is a lysochrome (fat-soluble dye) diazo dye used for staining of neutral triglycerides and lipids on frozen sections and some lipoproteins on paraffin sections. In histology, a supersaturated solution of Oil-red-O in isopropanol may be used to stain fat in tissue.

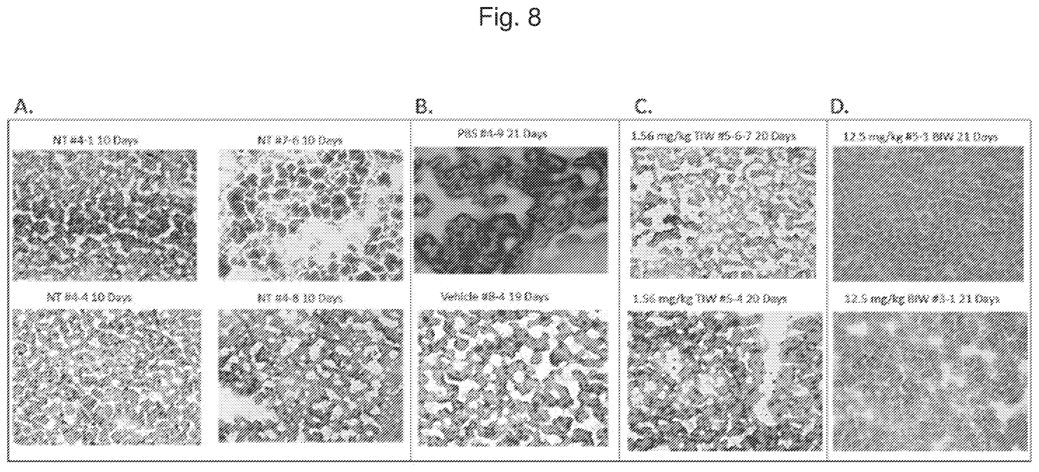

Liver samples collected from 10-day old untreated mice, 19 to 21-day old PBS treated mice, and 20 day old mice treated TIW with 1.56 mg/kg HIS-CGL-ILMDRGVS-PEG were stained with Oil-red-O. These mice all displayed greatly increased Oil-red-O staining compared to samples collected from 21-day old mice treated BIW with 12.5 mg/kg HTS-CGL-ILMDRGVS-PEG, indicating a reduction of neutral triglyceride, lipid, and/or lipoprotein in livers of mice dosed BIW with 12.5 mg/kg HIS-CGL-ILMDRGVS-PEG (FIGS. 8A, B, C, and D).

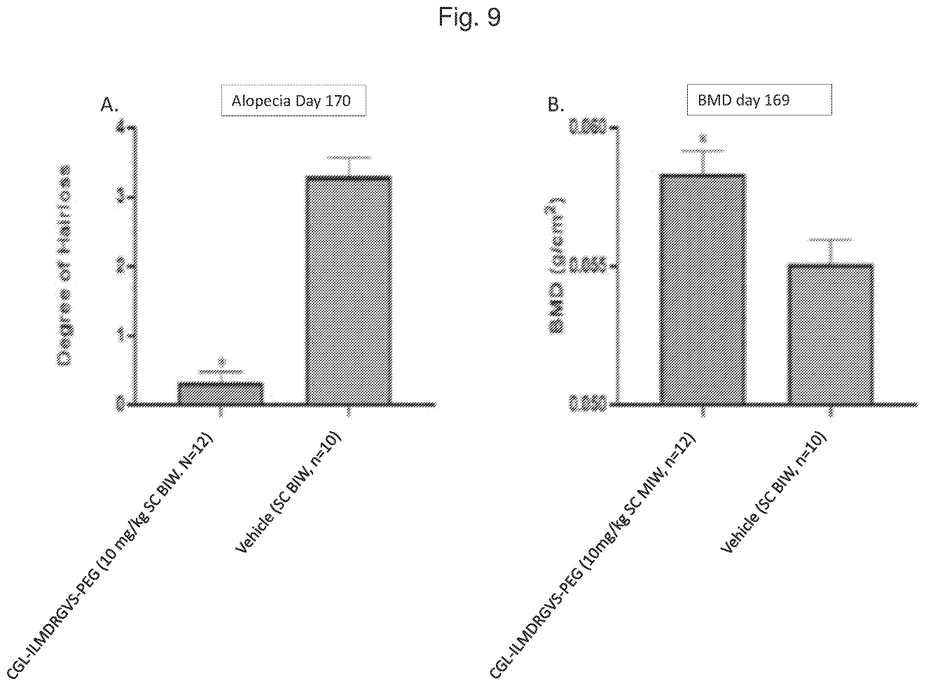

The effect on alopecia and osteoporosis was measured in CBS−/− mice SC treated BIW with 10 mg/kg CGL-ILMDRGVS-PEG. At day 170, the degree of hair loss significantly decreased_{[RA1]} in the CGL-ILMDRGVS-PEG treated CBS−/− mice compared to the vehicle control group (FIG. 9A). At day 169, the level of bone mineral density (BMD) was significantly higher in CGL-ILMDRGVS-PEG treated CBS−/− mice compared to the vehicle control group (FIG. 9B)._{[RA2]}

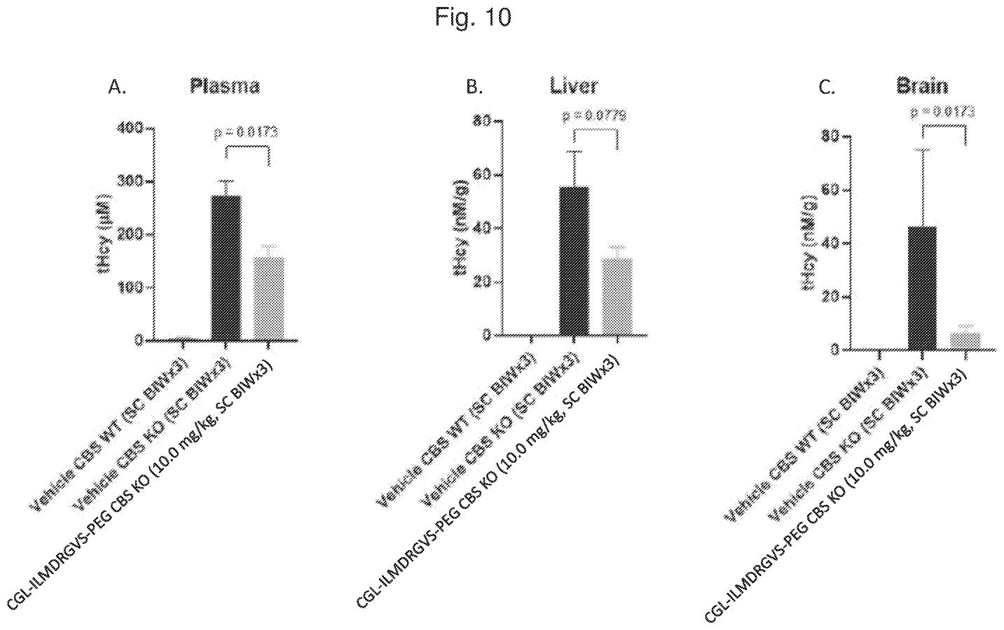

Total homocysteine (tHcy) was measured in plasma, liver and brain samples of vehicle-treated wild-type and CBS−/− mice, and CGL-ILMDRGVS-PEG treated CBS−/− mice (FIGS. 10A, B, and C). All mice were treated subcutaneously three times BIW. Vehicle-treated CBS−/− mice showed a significant increase in plasma, liver and brain tHcy levels compared to the wild-type group. Treatment in CBS−/− mice with 10 mg/kg CGL-ILMDRGVS-PEG significantly reduced tHcy levels in plasma, liver and brain samples versus the vehicle-treated group indicating efficacious effects of using engineered CGL as a potential therapy for homocystinuria.

The observed survival effect of HIS-CGL-ILMDRGVS-PEG in the CBS−/− mice was used to perform pharmacodynamic analyses. For the CBS−/− studies described in next sections. “maintenance” dosing of animals ranged between 5 and 10 weeks.

### Example 2: Single-Dose Pharmacodynamic Studies in CBS−/− Mice

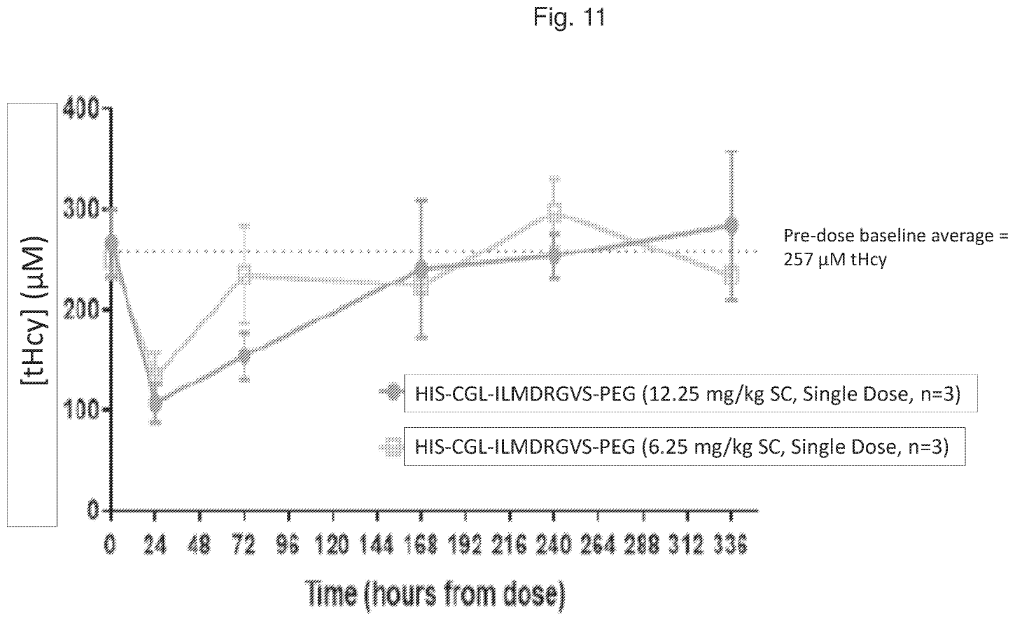

The pharmacodynamic (PD) effects of HIS-CGL-ILMDRGVS-PEG were assessed by administration to CBS−/− mice. CBS−/− mice are an animal model of human homocystinuria due to CBS deficiency that presents with elevated levels of plasma tHcy. CBS−/− mice that demonstrated survival over 12 weeks (and were therefore no longer at risk of neonatal lethality) were removed from HIS-CGL-ILMDRGVS-PEG dosing (multiple dose levels) and given a 25-day washout period to clear any residual drug and allow plasma tHcy levels to return to their abnormally high baseline level. During this time, the animals were closely monitored for deteriorating clinical signs resulting from increased circulating tHcy. The mice were then randomized into 2 groups and dosed a single time with HIS-CGL-ILMDRGVS-PEG at either 12.5 or 6.25 mg/kg subcutaneously (SC). Blood was collected at multiple timepoints over the course of 2 weeks. Initial concentrations of plasma tHcy were ˜250 μM and maximal inhibition for both groups (reduction to ˜100 PM) was observed at the first collected timepoint 24 hours after dosing. By 72 hours after dosing, the plasma tHcy levels in the 6.25 mg/kg dosed group returned to baseline levels where they remained for the remainder of the study. The plasma tHcy levels in the 12.5 mg/kg dosed group remained at ˜100 μM through 72 hours and returned to baseline values by 168 hours (FIG. 11).

Further analysis of (CBS−/− mice) PD showed pre-dose plasma tHcy levels averaged 257 μM and were reduced on average to 107.3 μM and 132.4 μM twenty-four hours after a single dose of HIS-CGL-ILMDRGVS-PEG at 12.5 or 6.25 mg/kg, respectively 72 hours after dosing, plasma tHcy returned to baseline in animals dosed with 6.25 mg/kg HIS-CGL-ILMDRGVS-PEG but remained at 153.7 μM in the 12.5 mg/kg dosed group (FIG. 11). 168 hours after dosing tHcy had returned to baseline in both groups.

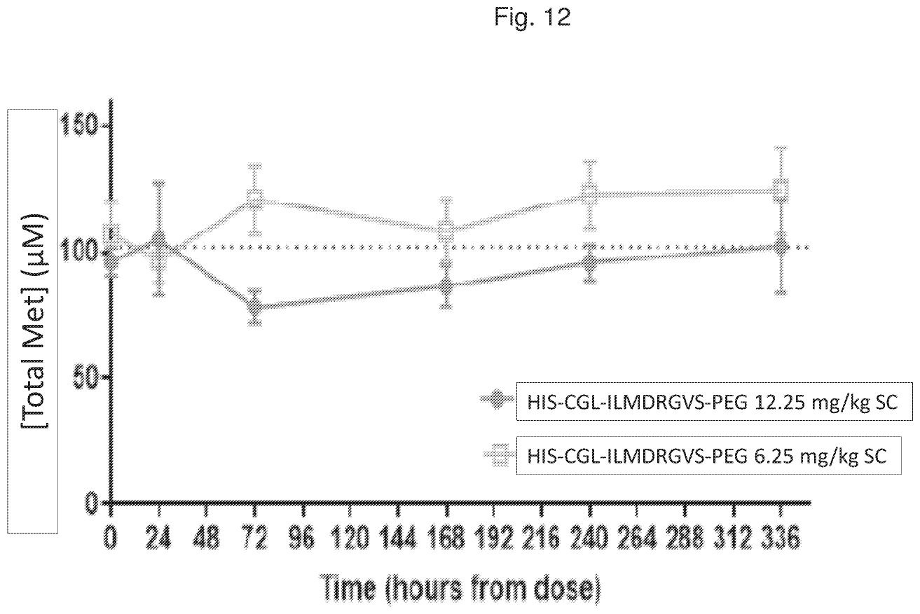

Pre-dose, CBS−/− mice methionine plasma levels were (on average) 102 μM. Dosing with 6.25 mg/kg did not decrease methionine levels throughout the 336-hour time course (FIG. 12). Seventy-two hours after dosing with 12.5 mg/kg HIS-CGL-ILMDRGVS-PEG plasma methionine levels were decreased to an average of 78 μM (24% decrease). By 168 hours methionine levels were at 82 uM and by 240 hours they returned to baseline (FIG. 12). Plasma total methionine levels following a single SC dose of 12.5 mg/kg or 6.25 mg/kg HIS-CGL-ILMDRGVS-PEG.

### Example 3: Multi-Dose Pharmacodynamic Studies in CBS−/− Mice

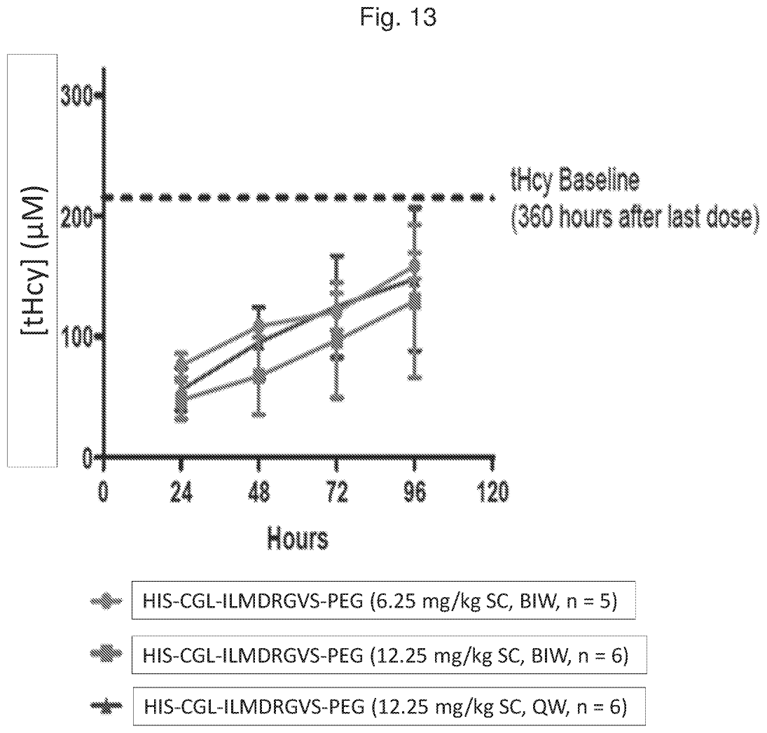

CBS−/− mice that demonstrated survival over 12 weeks were removed from HIS-CGL-ILMDRGVS-PEG dosing (multiple dose levels) and given a 15-day washout period to clear any residual drug and allow tHcy levels to return to their abnormally high baseline level. After the washout period, mice were randomized into 3 groups and dosed with HIS-CGL-ILMDRGVS-PEG at either 12.5 or 6.25 mg/kg SC twice per week (BIW) or 12.5 mg/kg once per week (QW) for 2 weeks. Due to blood volume constraints, animals were not bled on consecutive timepoints following multiple doses of drug. Instead, all animals received a minimum of 4 total doses of HIS-CGL-ILMDRGVS-PEG and blood was collected 24, 48, 72, or 96 hours after 8, 6, 4, or 8 doses, respectively, for the BIW-dosed groups and 5, 4, 3, or 2 doses, respectively for the QW-dosed group. Post washout starting levels of tHcy were ˜220 uM and maximal reduction for both groups was observed at 24 hours after dosing (reduction to ˜50 μM). All groups with repeat dosing maintained tHcy levels below 100 μM through 48 hours and the 12.5 mg/kg BIW-dosed groups had tHcy levels sustained below 100 μM through 72 hours (FIG. 13).

### Example 4: Intravenous (IV) Pharmacokinetics CGL-ILMDRGVS-PEG in Monkeys

The PK of CGL-ILMDRGVS-PEG was determined in non-naive male cynomolgus monkeys. The PK study had 2 parts: Phase 1 (single intravenous (IV) dose(s)) and Phase 2 (repeat subcutaneous (SC) dosing). In Phase 1, monkeys were administered CGL-ILMDRGVS-PEG by intravenous (IV) or subcutaneous (SC) injection on Day 1. The same animals that received initial SC injection were also proceeded to Phase 2 with a minimum 14-day washout period between dosing for each phase. Following an approximate 14-day washout period subsequent to Phase 1 dosing, each animal in Group 3 was dosed SC once weekly (QW) with CGL-ILMDRGVS-PEG at the appropriate dose level for three consecutive weeks (Table 4). In addition, each animal received a SC dose of the vehicle control, administered at a separate location and at the same volume and frequency as the test article. The study design is summarized in Table 4.

TABLE 4
Study design for Pharmacokinetics of
CGL-ILMDRGVS-PEG in Monkeys

| | | | | | |
|---|---|---|---|---|---|
| | | | | | Dose |
| | | | | Dose | Vol. |
| | Test | No. of | Dose | (mg/ | (mL/ |
| Group | Article | Males | Route | kg) | kg) |

PHASE 1 - SINGLE DOSE

| | | | | | |
|---|---|---|---|---|---|
| 1 | CGL-ILMDRGVS-PEG | 3 | IV | 2 | 0.4 |
| 2 | CGL-ILMDRGVS-PEG | 3 | IV | 8 | 1.6 |
| 3 | CGL-ILMDRGVS-PEG | 3 | SC | 8 | 1.6 |

PHASE 2 - REPEAT DOSE

| | | | | | |
|---|---|---|---|---|---|
| 3 | CGL-ILMDRGVS-PEG + | 3 | SC | 2 | 0.4 |
| | Vehicle Control | | | 0 | 0.4 |

The animals in Groups 1 and 2 received a single IV dose of CGL-ILMDRGVS-PEG. The animals in Groups 3 (Phase 1) received a single SC dose of CGL-ILMDRGVS-PEG. Male monkeys in Groups 1 and 2 were administered single IV dose(s) of CGL-ILMDRGVS-PEG at 2 and 8 mg/kg.

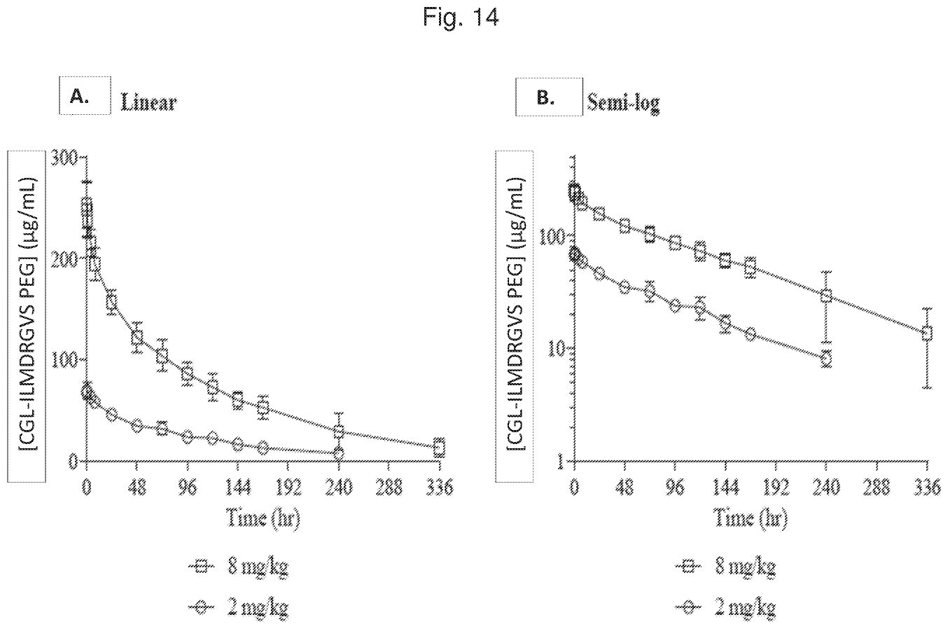

Increases in IV exposure (Cₘₐₓ, AUC₀₋₂₈) were close to dose proportional. Mean (±SD) Cₘₐₓ was 72.3±8.03 μg/mL at 2 mg/kg and 257±18.7 μg/mL at 8 mg/kg. Mean (±SD) AUC_{0-∞} was 7050±419 and 24100±4090 hr*g/mL, respectively. Thus, there were ˜3.6-fold and ˜3.4-fold increases in mean Cₘₐₓ and AUC0-∞ for a 4-fold change in dose. Median Tₘₐₓ was observed at 0.5 hours post dose (Table 5 and FIG. 14A, B).

TABLE 5
Mean (+SD) PK Parameters for CGL-ILMDRGVS-PEG in Male Monkeys
After a Single IV Dose

| | | | | | AUC₀₋ₜ | AUC₀₋₁₆₈ | AUC_{0-∞} | | |
|---|---|---|---|---|---|---|---|---|---|
| Dose | Stat. | T_{1/2} | Tₘₐₓ¹ | Cₘₐₓ | (hr * | (hr * | (hr * | CL | Vₛₛ |
| (mg/kg) | Param. | hr | hr | (μg/mL) | μg/mL) | μg/mL) | μg/mL | mL/hr/kg | mL/kg |
| 2 | N | 3 | 3 | 3 | 3 | 3 | 3 | 3 | 3 |
| | Mean | 93.6 | 0.500 | 72.3 | 5950 | 5190 | 7050 | 0.285 | 36.3 |
| | SD | 14.9 | 0.500, | 8.03 | 393 | 340 | 419 | 0.0165 | 4.40 |
| | | | 1.00 | | | | | | |
| | CV % | 15.9 | NA | 11.1 | 6.6 | 6.5 | 6.0 | 5.8 | 12.1 |
| 8 | N | 3 | 3 | 3 | 3 | 3 | 3 | 3 | 3 |
| | Mean | 88.4 | 0.400 | 25.7 | 22000 | 17700 | 24100 | 0.339 | 41.6 |
| | SD | 37.3 | 0.0833, | 18.7 | 3850 | 2040 | 4090 | 0.0629 | 9.85 |
| | | | 1.00 | | | | | | |
| | CV % | 42.2 | NA | 7.3 | 17.4 | 11.5 | 16.9 | 18.6 | 23.7 |

¹Median and range (Min, Max) presented;
NA-not applicable

Circulating concentrations of CGL-ILMDRGVS-PEG were higher at 8 mg/kg and were measurable for a longer time relative to the circulating concentrations at 2 mg/kg (FIG. 14). Mean clearance (CL) appeared slightly lower at the lower dose but the ranges (mean±SD) were overlapping (Table 5). Mean (±SD) CL was 0.285±0.0165 mL/hr/kg at 2 mg/kg and 0.339±0.0629 mL/hr/kg at 8 mg/kg. The observed volume of distribution (Vₛₛ) was mostly comparable to monkey serum volume (45 mL/kg) although the mean estimates were trending a bit lower. Mean Vₛₛ (±SD) was 36.3±4.40 mL/kg at 2 mg/kg and 41.6±9.85 mL/kg at 8 mg/kg. The resulting mean half-life (T_{1/2}) estimates ranged from 88.4 to 93.6 hr (˜3.7-3.9 days).

Increases in IV exposure (Cₘₐₓ, AUC_{0-∞}) were close to dose proportional (Table 5). Mean (±SD) Cₘₐₓ was 72.3±8.03 ag/mL at 2 mg/kg and 257±18.7 μg/mL at 8 mg/kg. Mean (±SD) AUC_{0-∞} was 7050±419 and 24100±4090 hr*μg/mL, respectively. Thus, there were ˜3.6-fold and ˜3.4-fold increases in mean Cₘₐₓ and AUC_{0-∞} for a 4-fold change in dose. Median Tₘₐₓ wats observed at 0.5 hr post dose.

### Example 5: Pharmacokinetics of Subcutaneous (SC) CGL-ILMDRGVS-PEG in Monkeys

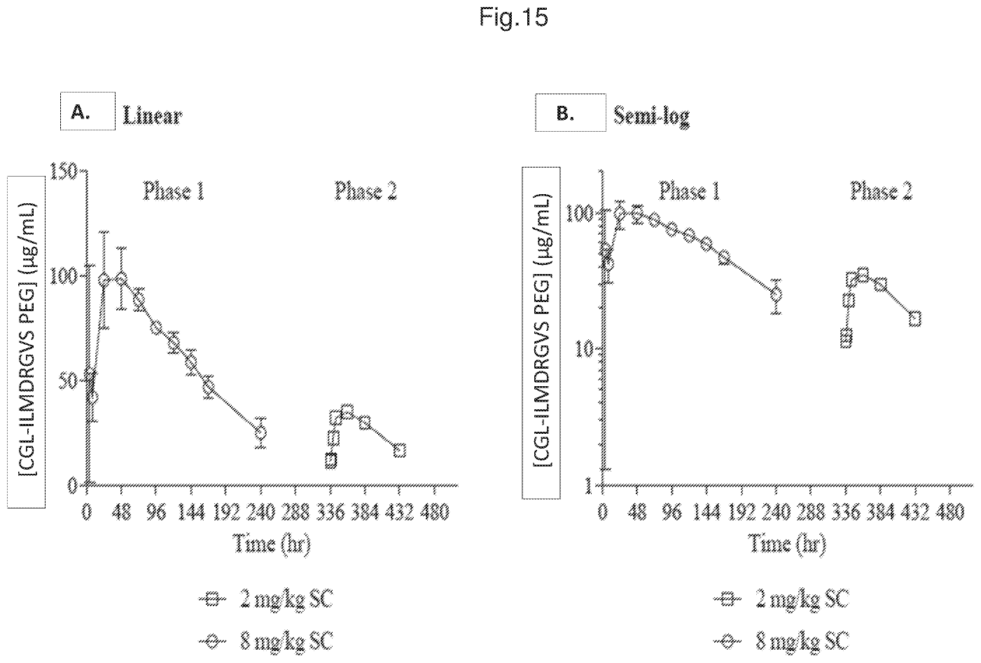

The mean PK parameters in male monkeys following repeat SC dose(s) of CGL-ILMDRGVS-PEG on Days 1 and 15 are summarized in Table 6. Mean serum concentration versus time profiles are presented in FIG. 15.

TABLE 6
Mean (±SD) Pk Parameters for CGL-ILMDRGVS-PEG in Male Monkeys After

[symbol]

| | | AUC₀₋ₜ | AUC₀₋₁₆₈ | AUC_{0-∞} | | | | | | | |
|---|---|---|---|---|---|---|---|---|---|---|---|
| Tₘₐₓ¹ | Cₘₐₓ | (hr * | (hr * | (hr * | CL/F² | V_{Z}/F² | F | Dose | | Stat. | T_{1/2} |
| hr | (μg/mL) | μg/mL) | μg/mL) | μg/mL | mL/hr/kg | mL/kg | (%) | (mg/kg) | Day | Param. | hr |
| 3 | 3 | 3 | 3 | 3 | 3 | 3 | 3 | 8 | 1 | N | 3 |
| 48.0 | 106 | 14900 | 12500 | 18.30 | 0.440 | 53.4 | 75.8 | | | Mean | 85.5 |
| 24.0, 48.0 | 17.7 | 792 | 1120 | 15.20 | 0.0384 | 13.3 | 7.63 | | | SD | 27.0 |
| NA | 16.7 | 5.3 | 9.0 | 8.3 | 8.7 | 24.9 | 10.1 | | | CV % | 31.5 |
| 2 | 2 | 2 | 1 | 1 | | | | 2 | 15 | N | 1 |
| 24.0 | 34.9 | 34.9 | 4710 | 7220 | | | | | | Mean | 106 |
| 24.0, 24.0 | NR | NR | NR | NR | | | | | | SD | NR |
| NA | NR | NR | NR | NR | | | | | | CV % | NR |

¹Median and range (Min, Max) presented;
²CL/F an VZ/F not reported on Day 15 as it was improbable that steady state had been achieved at the time of the 2^{nd} lower dose.

[symbol]

[symbol]

indicates data missing or illegible when filed

[symbol]

Male monkeys were administered two repeat SC injection(s) of CGL-ILMDRGVS-PEG at 8 (1st dose) and 2 (2nd dose) mg/kg, with a 2-week washout between doses. Circulating mean concentrations of CGL-ILMDRGVS-PEG were higher at 8 mg/kg and were measurable for a longer time relative to the circulating concentrations at 2 mg/kg (FIG. 15). Consequently, SC exposure (Cₘₐₓ, AUC_{0-∞}) was higher at 8 vs. 2 mg/kg (Table 6). Mean (±SD) Cₘₐₓ was 106±17.7 μg/mL at 8 mg/kg and 34.9 (N=2) μg/mL at 2 mg/kg. Mean (±SD) AUC_{0-∞} was 18,300±1520 and 4710 (N=1) hr*g/mL, respectively.

Median Tₘₐₓ was later for the higher 8 mg/kg dose at 48 hour vs. 24 hour at 2 mg/kg (Table 6). The mean bioavailability estimate at 8 mg/kg was moderate at 75.8%. The mean extravascular CL (CL/F) estimate was slightly higher than the IV CL estimate at 8 mg/kg (e.g., 0.440±0.0384 vs. 0.339±0.0629 mL/kg/kg, respectively; Table 5 and Table 6), consistent incomplete absorption.

### Example 6: Comparison of IV and SC Pharmacodynamics

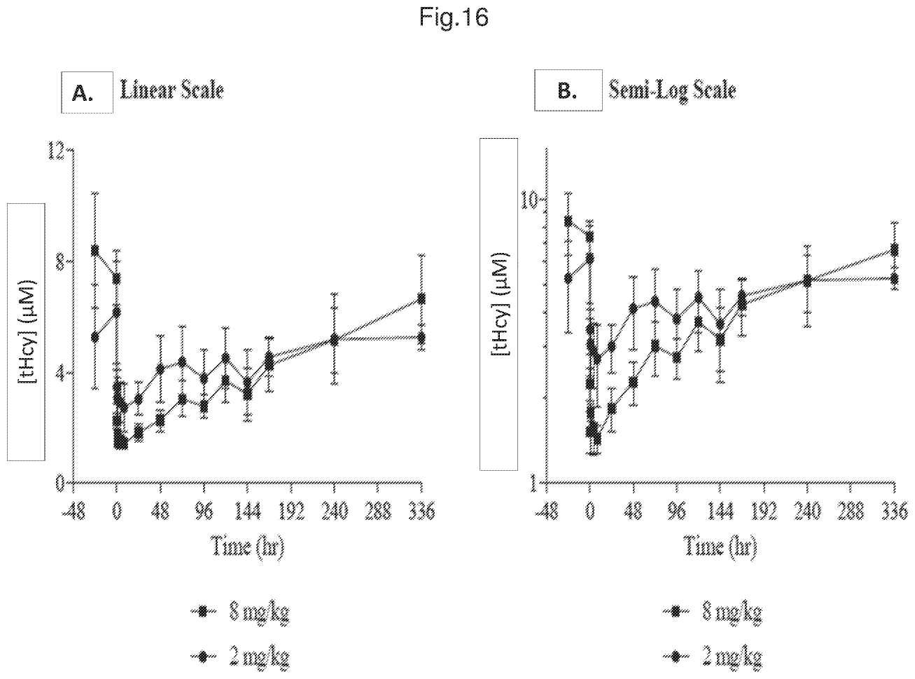

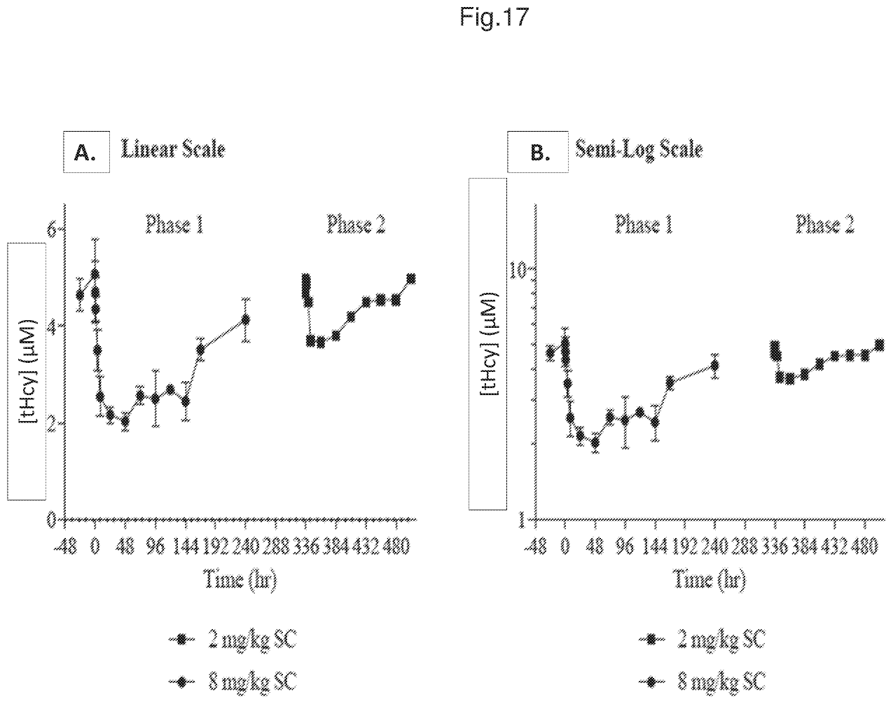

Mean concentration versus time profiles for tHcy levels in plasma after IV and SC administration of CGL-ILMDRGVS-PEG are presented in FIG. 16 and FIG. 17, respectively.

Following IV administration of CGL-ILMDRGVS-PEG, maximal reduction of plasma tHcy levels was observed at 4-8 hours post dose and more mean reduction was observed for the higher IV dose level of 8 mg/kg (FIG. 16). There was an initial ˜39% and ˜72% drop in plasma tHcy levels at 5 minutes post dose (at 2 and 8 mg/kg respectively), followed by a slower decline to the maximal observed reduction. At 8 hours post dose, mean tHcy levels were at 47.8% and 18.1% of the average baseline levels (corresponding to 52.2% and 81.9% decreases) for the 2 and 8 mg/kg dose groups, respectively. This corresponded to mean CGL-ILMDRGVS-PEG concentrations at or above 58.6 and 194 μg/mL, respectively. Recovery to baseline tHcy levels was mostly complete by 240 hours (˜91%) for the 2 mg/kg IV dose group and ˜84% complete by 336 hours (2 weeks) for the 8 mg/kg IV dose group.

Following SC administration of CGL-ILMDRGVS-PEG, maximal reduction of plasma tHcy levels was observed at 24-48 hours post dose at the 8 mg/kg dose level and at 8-24 hours for the 2 mg/kg dose (FIG. 17). In contrast to IV administration, after dosing there was a steady decline in tHcy levels until maximal reduction was achieved. At maximal reduction, mean tHcy levels were at 75.5% and 42.0% of the average baseline levels (corresponding to 24.5% and 58.0% decreases) for the 2 and 8 mg/kg dose levels, respectively, noting that 2 mg/kg CGL-ILMDRGVS-PEG was administered 2 weeks after 8 mg/kg CGL-ILMDRGVS-PEG (in the same animals). This corresponded to mean CGL-ILMDRGVS-PEG concentrations up to 34.9 and 98.7 μg/mL, respectively, noting that there was marked inter-animal PK variability at 2 mg/kg. Recovery to baseline tHcy levels was complete (102%) by 336 hours or Day 15 predose (2 weeks) for the 8 mg/kg/Day 1 dose. Following the 2 mg/kg dose on Day 15, the recovery appeared complete by 168 hours post dose (102%).

### Example 7: Clinical Development Plan: First-in-Human Phase 1/2 Multiple Ascending-Dose Study

The purpose of a Phase 1/2 multiple ascending-dose study is to investigate the safety, pharmacokinetics, and pharmacodynamics of CGL-ILMDRGVS-PEG in subjects with homocystinuria due to cystathionine β-synthase (CBS) deficiency. For the first-in-human study, there is no randomization or blinding, and the study is open-label. The primary, secondary, and exploratory objectives along with their corresponding endpoints are provided in Table 7.

TABLE 7
Clinical Study Objectives and Endpoints

| | |
|---|---|
| Objectives | Endpoints |

Primary

| | |
|---|---|
| Evaluate the safety and | Incidence of treatment-emergent adverse |
| tolerability of CGL- | events (TEAEs) |
| ILMDRGVS-PEG in | |
| subjects | |

Secondary

| | |
|---|---|
| Characterizing the | Determining the PK parameters and PK/PD |
| pharmacokinetic (PK) and | relationship after CGL-ILMDRGVS-PEG |
| pharmacodynamic (PD) | administration: |
| relationship of CGL- | Time to maximum observed concentration |
| ILMDRGVS-PEG after | (tₘₐₓ) |
| single and multiple doses | Maximum observed concentration (Cₘₐₓ), |
| following intravenous (IV) | area under the concentration-time curve |
| and subcutaneous (SC) | through the time of the last measurable |
| administration | concentration (AUC₀₋ₜ) |
| | Area under the concentration-time curve |
| | extrapolated to infinity (AUC_{0-∞}) |
| | Apparent terminal elimination half-life |
| | (t_{1/2}) |
| | Serum clearance of drug per unit time (CL |
| | after IV dosing; CL/F after SC dosing) |
| | Volume of distribution (Vₛₛ after IV |
| | dosing; V_{z}/F after SC dosing) |
| Evaluating the onset, | Determining the proportion of subjects |
| magnitude of change, | who achieve target plasma tHcy of ≤50 |
| and reversibility of | μM after treatment with CGL- |
| changes in plasma total | ILMDRGVS-PEG |
| homocysteine (tHcy) | Determining proportion of subjects who |
| | achieve target plasma tHcy of ≤15 μM |
| | after treatment with CGL-ILMDRGVS- |
| | PEG |

Exploratory

| | |
|---|---|
| Examining the impact | Compare biomarker and plasma amino acid |
| of CGL-ILMDRGVS- | levels before and after administration of |
| PEG on biomarkers and | CGL-ILMDRGVS-PEG |
| plasma amino acids | Compare consistency of diet and dietary |
| Examining the burden | restrictions via the 3-day diet record and |
| of dietary restriction | the individualized diet management |
| in subjects with | questionnaire before and after |
| homocystinuria due | administration of CGL-ILMDRGVS-PEG |
| to CBS deficiency | |
| Determining baseline | Characterize baseline: |
| neurocognitive | neurocognitive function via Wechsler |
| function, quality of | Adult Intelligence Scale IV (WAIS-IV), |
| life (QoL), and | including the General Ability Index (GAI) |
| overall subject health | and the Wechsler Intelligence Scale for |
| relative to reference | Children V (WISC-V) for subjects ≥12 |
| populations | but <16 years of age |
| | Quality of life (QoL) by the 36-Item Short |
| | Form Health Survey (SF-36) (subjects ≥19 |
| | years of age) and the Pediatric Quality |
| | of Life Inventory (PedsQL) (subjects 2 |
| | to <19 years of age) |
| | Overall subject health via routine |
| | laboratory assessments and physical |
| | examinations |

Subjects are eligible to be included in the study if all of the following criteria apply: (i) diagnosis of homocystinuria due to CBS deficiency, (ii) capable of providing signed informed consent/assent, which includes compliance with the requirements and restrictions listed in the informed consent form (ICF) and in this protocol, (iii) is ≥12 years of age at the time of signing the informed consent/assent, (iv) plasma tHcy>80 uM on at least 1 of the screening visits, (v) female subjects of child-bearing potential must have a negative serum pregnancy test during the screening period before receiving the first dose of study drug and a negative urine pregnancy test prior to dosing on the first day of treatment (vi) if the subject (male or female) is engaging in sexual activity, he/she must be unable to become/cause pregnancy or must agree to use highly effective contraception, as specified in the full protocol, and (vii) subjects receiving pyridoxine and/or betaine must be on a stable dose of the medication(s) for at least 6 weeks prior to the first administration of study drug and be willing and able to remain on a stable dose for the duration of the study.

Subjects are excluded from the study if any of the following criteria apply: (i) other medical conditions or co-morbidity(ies) that, in the opinion of the investigator, would interfere with study compliance or data interpretation (e.g., severe intellectual disability that precludes completion of the required study assessments), (ii) currently participating in another therapeutic clinical study or has received any investigational agent within 30 days or 5 half-lives, whichever is longer, prior to the first dose of study drug in this study, (iii) surgery requiring general anesthesia within the 8 weeks prior to first dose of study drug, (iv) active infection requiring anti-infective therapy <2 weeks prior to the first dose of study drug in this study; anti-infective therapy that completes ≥2 weeks prior to first dose of study drug is acceptable, (v) pregnant or nursing, (vi) females of child-bearing potential who are using or plan to use estrogen-containing contraception during the study, and (vii) history of hypersensitivity to polyethylene glycol (PEG) that, in the judgment of the investigator, puts the subject at unacceptable risk for adverse events (AEs).

For cohort enrollment, each cohort will begin with the dosing of a sentinel subject. If after at least 48 hours of post-dose monitoring of the sentinel subject, CGL-ILMDRGVS-PEG is deemed safe and to have an acceptable tolerability profile, then subsequent subjects can be dosed.

Part 1: IV Dosing

4 subjects in in cohort 1 will receive a dose of 0.15 mg/kg of CGL-ILMDRGVS-PEG administered by intravenous (IV) infusion over approximately 30 minutes. A total of up to 4 doses given once-weekly (QW) will be administered to subjects in all cohorts. After 4 doses of CGL-ILMDRGVS-PEG are administered to each of the first 2 subjects in Part 1, safety data, plus all available PK and PD data will be reviewed. If no stopping rules are met, transition to Part 2 will occur.

Part 2: SC Dosing

Subjects in cohort 2 will receive a dose of 0.15 mg/kg of CGL-ILMDRGVS-PEG administered by subcutaneous (SC) injection. Assuming stopping rules are not met, subjects in the second cohort will receive a dose of 0.45 mg/kg, and subjects in the third cohort will receive a dose of 1.0 mg/kg. A total of up to 4 doses given QW will be administered to subjects in all cohorts. If an additional cohort is added, the dose will be determined based on the data from the previous 3 cohorts and the dose can be no more than 2-fold higher than the highest dose tested if, following review of all safety, PK, and PD data from all planned cohorts, all of the following apply: (i) safety stopping rules for dose escalation and study termination are not met and (ii) the plasma tHcy taken 168 hours after Dose 4 in 1 or more subjects in SC Cohort 3 (highest planned dose) is >4 μM (the lower limit of normal [LLN]).

Stopping rules and exception for the study are described in Table 8.

TABLE 8
Stopping Rules and Exceptions for First-in-Human Phase 1/2
Multiple Ascending-Dose Study

| Condition | Occurrence |
|---|---|
| Enrollment and dosing will be | 2 subjects develop the same Grade 3 |
| temporarily halted, a safety | treatment-rated AE |
| review completed, and a | 1 subject develops a ≥ Grade 4 treatment- |
| determination made to | related AE |
| permanently stop, modify, or | A ≥ Grade 3 treatment-related AE |
| continue the study if any of the | |
| following occur: | |
| Enrollment to a dose cohort | ≥2 subjects develop the same Grade 2 |
| will be halted, a safety review | treatment-rated AE that lasts longer than 3 |
| completed, and a determination | days despite appropriate intervention |
| made to proceed with dose | ≥1 subject develop the same Grade 3 |
| escalation, continue enrollment | treatment-rated AE |
| to current cohort, or | |
| permanently stop enrollment | |
| if any of the following occur: | |
| To prevent unnecessary early | Hypersensitivity/Allergic Reactions: If study |
| termination of a subject, dose | drug is being administered IV, the infusion |
| escalation, or the study, the | will be interrupted in any subject who |
| following exceptions will | develops signs or symptoms of |
| apply to consideration of | hypersensitivity. The infusion may be |
| the stopping rules: | resumed if the symptoms can be safely |
| | managed. The hypersensitivity/allergic |
| | reaction, regardless of route of |
| | administration, will be treated according to |
| | standard of care. A manageable reaction is |
| | not automatic cause for stopping dosing in |
| | an individual subject or dose escalation in |
| | the study. Subsequent doses may be |
| | administered and pre-medication with anti- |
| | histamines an anti-pyretics is recommended |
| | prior to all remaining study drug |
| | administration. Corticosteroids are allowed |
| | according to the local policy. |
| | Baseline and disease-related AEs: An AE |
| | that was present at baseline or is a |
| | manifestation of the disease under treatment |
| | will be subject to the stopping rules only if |
| | the TEAE exhibits an increase in ≥1 from |
| | baseline. |

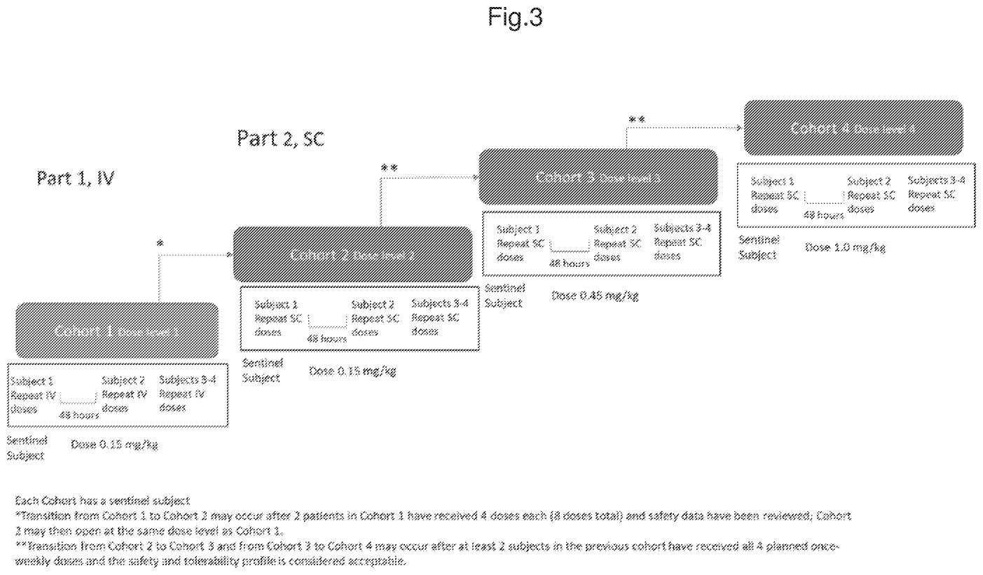

A schematic for the first-in-human Phase 1/2 multiple ascending-dose study to test CGL-ILMDRGVS-PEG is provided FIG. 3.

Reference throughout this specification to “one embodiment,” “certain embodiments,” “various embodiments,” “one or more embodiments” ˜ or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in various embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein provided a description with reference to particular embodiments, it is to be understood that these embodiments are merely illustrative of the principles and applications of the disclosure. It will be apparent to those skilled in the art that various modifications and variations can be made to the present disclosure without departing from the spirit and scope thereof. Thus, it is intended that the present disclosure include modifications and variations that are within the scope of the appended claims and their equivalents.

What is claimed is:

1. A method of treating a subject having or at risk of developing homocystinuria or hyperhomocysteinemia, the method comprising:
administering a therapeutically effective amount of formulation comprising a modified human cystathionine-γ-lyase (CGL) enzyme comprising at least the following substitutions relative to a native human CGL amino acid sequence (SEQ ID NO: 1): isoleucine at position 59, leucine at position 63, methionine at position 91, aspartic acid at position 119, arginine at position 268, glycine at position 311, valine at position 339, and serine at position 353;
wherein the formulation is administered at an enzyme dose of about 0.05 mg/kg to about 4 mg/kg at a frequency of one dose per day to one dose per month.

2. The method of claim 1, wherein the enzyme is bound to one or more PEG units.

3. The method of claim 1 or 2, wherein the formulation further comprises a pharmaceutically acceptable carrier.

4. The method of any one of claims 1-3, wherein the formulation is 2 mL of a liquid supplied in a 5 mL vial.

5. The method of any one of claims 1-4, wherein the formulation comprises an enzyme concentration of about 1 mg/mL to about 50 mg/mL.

6. The method of any one of claims 1-5, wherein the formulation comprises an enzyme concentration of about 1 to about 20 mg/mL.

7. The method of any one of claims 1-6, wherein the formulation is administered intravenously or subcutaneously.

8. The method of any one of claims 1-7, wherein the formulation is administered one dose per week.

9. The method of any one of claims 1-8, wherein the formulation is administered intravenously for four weeks, followed by subcutaneous administration in subsequent weeks.

10. The method of any one of claims 1-9, wherein the formulation is administered to the subject at a dose of about 0.1 mg/kg to about 1 mg/kg.

11. The method of any one of claims 1-10, wherein the formulation is administered once a week at a dose of about 0.1 mg/kg to about 1 mg/kg.

12. The method of any one of claims 1-11, wherein the formulation is administered to the subject at a dose of about 0.15 mg/kg, about 0.45 mg/kg, or about 1 mg/kg.

13. The method of any one of claims 1-12, wherein the formulation is diluted in saline prior to intravenously administering to the subject.

14. The method of any one of claims 1-13, wherein the subject is a human patient.

15. The method of any one of claims 1-14, wherein the subject has a total plasma homocysteine level greater than 80 μM prior to initiating therapy with administering the formulation.

16. The method of any one of claims 1-15, wherein the subject is at least 12 years of age.

17. The method of any one of claims 1-16, wherein the subject is maintained on an individualized diet.

18. The method of any one of claims 1-17, wherein the subject is maintained on a methionine-restricted diet.

19. The method of any one of claims 1-18, wherein the method reduces total plasma homocysteine levels.

20. The method of any one of claims 1-19, wherein the method reduces total plasma homocysteine levels to less than or equal to about 80 μM.

21. The method of any one of claims 1-20, wherein the method reduces total plasma homocysteine levels to less than or equal to about 50 μM.

22. The methods of any one of claims 1-21, wherein the method reduces total plasma homocysteine levels to less than or equal to about 15 uM.
